SEQUENCE LISTING (22 sequences)

SEQ ID NO: 1 - protein, 20 aa, Artificial sequence
  Synthetic peptide
HRKKRRQRRITRKKTFKEVA

SEQ ID NO: 2 - protein, 1203 aa, Homo sapiens
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDDPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

SEQ ID NO: 3 - protein, 20 aa, Homo sapiens
TRKKTFKEVANAVKISASLM

SEQ ID NO: 4 - protein, 11 aa, Artificial sequence
  Synthetic peptide
ITRKKTFKEVA

SEQ ID NO: 5 - protein, 4 aa, Artificial sequence
  Synthetic peptide
  misc_feature (1)..(1): Xaa can be any naturally occurring amino acid
  misc (2)..(2): Can be T
  misc_feature (3)..(3): Xaa can be any naturally occurring amino acid
  misc (4)..(4): Can be K
XSXR

SEQ ID NO: 6 - protein, 4 aa, Artificial sequence
  Synthetic peptide
KTFK

SEQ ID NO: 7 - protein, 10 aa, Artificial sequence
  Synthetic peptide
TRKKTFKEVA

SEQ ID NO: 8 - protein, 9 aa, Artificial sequence
  Synthetic peptiide
RKKRRQRRR

SEQ ID NO: 9 - protein, 8 aa, Artificial sequence
  Synthetic peptiide
RRRRRRRR

SEQ ID NO: 10 - protein, 16 aa, Artificial sequence
  Synthetic peptiide
RKKRRRESRKKRRRES

SEQ ID NO: 11 - protein, 17 aa, Artificial sequence
  Synthetic peptiide
GRPRESGKKRKRKRLKP

SEQ ID NO: 12 - protein, 16 aa, Artificial sequence
  Synthetic peptiide
RQIKIWFQNRRMKWKK

SEQ ID NO: 13 - protein, 13 aa, Artificial sequence
  Synthetic peptiide
GRRRRRRRRRPPQ

SEQ ID NO: 14 - protein, 18 aa, Artificial sequence
  Synthetic peptiide
LLIILRRRIRKQAHAHSK

SEQ ID NO: 15 - protein, 13 aa, Artificial sequence
  Synthetic peptiide
RVRVFVVHIPRLT

SEQ ID NO: 16 - protein, 27 aa, Artificial sequence
  Synthetic peptiide
GALFLGFLGAAGSTMGAWSQPKKKRKV

SEQ ID NO: 17 - protein, 18 aa, Artificial sequence
  Synthetic peptiide
KLALKLALKALKAALKLA

SEQ ID NO: 18 - protein, 27 aa, Artificial sequence
  Synthetic peptiide
GWTLNSAGYLLGKINLKALAALAKKIL

SEQ ID NO: 19 - protein, 5 aa, Artificial sequence
  Synthetic peptiide
VSALK

SEQ ID NO: 20 - protein, 16 aa, Artificial sequence
  Synthetic peptiide
CSIPPEVKFNPFVYLI

SEQ ID NO: 21 - protein, 26 aa, Artificial sequence
  Synthetic peptiide
GIGAVLKVLTTGLPALISWIKRKRQQ

SEQ ID NO: 22 - protein, 20 aa, Artificial sequence
  Synthetic peptide
HGLASTLTRWAHYNALIRAF

(19) United States
(12) Patent Application Publication
(10) Pub. No.: US 2024/0002437 A1
(43) Pub. Date: Jan. 4, 2024

(54) COMPOSITIONS AND METHODS OF TREATING INFLAMMATORY LUNG DISEASES

(71) Applicant: Arizona Board of Regents on Behalf of the University of Arizona, Tucson, AZ (US)

(72) Inventors: Stephen M. Black, Tucson, AZ (US); Evgeny A. Zemskov, Tucson, AZ (US); Qing Lu, Tucson, AZ (US)

(21) Appl. No.: 18/251,078

(22) PCT Filed: Oct. 25, 2021
(86) PCT No.: PCT/US2021/056440

§ 371 Date: Apr. 28, 2023

Related U.S. Application Data
(60) Provisional application No. 63/108,157, filed on Oct. 30, 2020.

(51) Int. Cl. C07K 5/113; A61P 11/00

(52) U.S. Cl. CPC C07K 5/1021; A61P 11/00; A61K 38/00

(57) ABSTRACT

It has been discovered that phosphorylation-dependent uncoupling of endothelial nitric oxide synthase (eNOS) plays an important role in endothelial cell (EC) barrier disruption. Compositions and methods to reduce or prevent eNOS uncoupling are disclosed. Decoy peptides that can prevent phosphorylation and mitochondrial redistribution of eNOS, reduce eNOS uncoupling, and preserve EC barrier function, and uses thereof, are described. The peptides improve lung vascular integrity in a mouse model of VILI. Thus, the decoy peptides can be used to treat or prevent diseases or disorders associated with increased vascular permeability such as ALI, ARDS, and VILI.

### CROSS REFERENCE TO RELATED APPLICATIONS

This application claims the benefit of and priority to U.S. Provisional Application No. 63/108,157 filed Oct. 30, 2020, which is hereby incorporated herein by reference in its entirety.

### STATEMENT REGARDING FEDERALLY SPONSORED RESEARCH

This invention was made with government support under Grant Nos. HL134610 and HL142212 awarded by National Institutes of Health. The government has certain rights in the invention.

### FIELD OF THE INVENTION

This invention is generally directed to compositions and the methods of use thereof to treat and prevent diseases associated with disruption of the endothelial barrier, including inflammatory lung disease and injury.

### BACKGROUND OF THE INVENTION

Mechanical ventilation is a life-saving intervention in critically ill patients with respiratory failure due to acute respiratory distress syndrome (ARDS), a refractory lung disease with an unacceptable high mortality (30-50%) (C. H. Goss, Crit Care Med, 31(6), 1607-11 (2003); M. A. Matthay, Am J Respir Crit Care Med, 167(7), 1027-35 (2003)). Paradoxically, mechanical ventilation also creates excessive mechanical stress that directly augments lung injury, a syndrome known as ventilator-induced lung injury (VILI) (C. H. Goss, Crit Care Med, 31(6), 1607-11 (2003); M. A. Matthay, Am J Respir Crit Care Med, 167(7), 1027-35 (2003); R. G. Brower, Tidal Volume Reduction, Critical Care Clinics, 18(1), 1-13 (2002); L. B. Ware, N Engl J Med, 342(18), 1334-49 (2000)). The deleterious synergy between excessive mechanical ventilation and ARDS, with a mortality of 30-40%, was underscored by the landmark ARDSnet trial (ARDSNet, N Engl J Med, 342(18), 1301-8 (2000)) with ARDS survival negatively influenced by mechanical ventilation-generated mechanical stress (C. H. Goss, Crit Care Med, 31(6), 1607-11 (2003); M. A. Matthay, Am J Respir Crit Care Med, 167(7), 1027-35 (2003); R. G. Brower, Tidal Volume Reduction, Critical Care Clinics, 18(1), 1-13 (2002)). VILI may also occur in mechanically-ventilated patients even when ARDS is not initially present (O. Gajic, Intensive Care Med, 31(7), 922-6 (2005)) and shares pathobiological features with ARDS including increased nuclear factor (NF)-κB-dependent inflammatory cytokine expression and marked lung endothelial cell (EC) protein leakage (D. P. Carlton, J Appl Physiol, 69(2), 577-83 (1990); D. Dreyfuss, Am J Respir Crit Care Med, 157(1), 294-323 (1998); D. Dreyfuss, The American Review of Respiratory Disease, 137(5), 1159-64 (1988); J. C. Parker, Am Rev Respir Dis, 142(2), 321-8 (1990); J. C. Parker, Crit Care Med, 21(1), 131-43 (1993); H. H. Webb, The American Review of Respiratory Disease, 110(5), 556-65 (1974)). The specific mechanisms involved in the development of VILI remain elusive highlighting the need for a more thorough understanding of VILI pathobiology and development of novel therapeutic targets and strategies.

Standard of care for ALI/ARDS uses protective lung ventilation strategies. However, these protective ventilation strategies are supportive and not therapeutic. Thus, there is intense interest in understanding the molecular mechanisms underlying VILI and ARDS in order to develop new treatment options. New prophylactic and therapeutic modalities are urgently needed to mitigate the public health, economic and societal impacts of VILI and ARDS, and in particular, to reduce morbidity and mortality associated therewith.

Therefore, it is an object of the invention to provide compositions and methods of use thereof for reducing and reversing the pathophysiological processes associated with the onset and progression increased vascular permeability.

It is an object of the invention to provide improved methods for treating lung injury or inflammatory lung disease to reduce severity or duration.

It is a further object of the invention to provide pharmaceutical compositions, and methods of use thereof, to prevent or treat diseases associated with increased vascular permeability such as ALI and ARDS.

### SUMMARY OF THE INVENTION

It has been discovered that endothelial nitric oxide synthase (eNOS) uncoupling plays an important role in the barrier disruption associated with ventilator induced lung injury (“VILI”). Studies demonstrate that pulmonary arterial endothelial cell (EC) barrier disruption is induced through the disruption of mitochondrial bioenergetics. Mechanistically, this occurs via PKC-dependent phosphorylation of eNOS at Threonine 495 (T495) leading to the mitochondrial redistribution of eNOS, followed by increased reactive oxygen species generation, and decreased mitochondrial membrane potential. A decoy peptide can prevent T495 phosphorylation and mitochondrial redistribution of eNOS, reduce eNOS uncoupling and preserve EC barrier function. Further, the eNOS decoy peptide preserved lung vascular integrity in a mouse model of VILI. Given this functional link between PKC-dependent eNOS phosphorylation at T495 and EC barrier permeability, reducing pT495-eNOS via decoy peptides is a therapeutic approach for the prevention, management or treatment of VILI and other vascular permeability related diseases.

Thus, compositions of synthetic peptides that can serve as eNOS decoys and methods of use thereof are provided. In particular, disclosed is an isolated, synthetic peptide having about 4 to 30 amino acids, and which can bind to or be bound by to Protein Kinase C (PKC), either in vitro or in vivo. In some embodiments, the peptide minimally includes a Protein Kinase C consensus binding sequence, such as X-S/T-X-R/K (SEQ ID NO:5), wherein X is any amino acid. An exemplary PKC consensus binding sequence is KTFK (SEQ ID NO:6). The peptide can further include from about 1-26 amino acids in addition to the consensus sequence. In some embodiments, the 1-26 additional amino acids constitute a functional peptide or domain, for example, a cell penetrating peptide.

In some embodiments, the peptide can be phosphorylated by Protein Kinase C (PKC), such as at a threonine residue. In preferred embodiments, the peptide includes the amino acid sequence HRKKRRQRRITRKKTFKEVA (SEQ ID NO:1) or ITRKKTFKEVA (SEQ ID NO:4), or an amino acid sequence having at least 70% sequence identity to HRKKRRQRRITRKKTFKEVA (SEQ ID NO:1) or ITRKKTFKEVA (SEQ ID NO:4).

In some embodiments, upon contacting the peptide with a cell or exposing a cell to the peptide, this reduces or prevents phosphorylation of endothelial nitric oxide synthase (eNOS) in the cell. For example, the phosphorylation of eNOS at threonine 495 (T495) can be reduced or prevented. Typically, the phosphorylation that is reduced or prevented is mediated by Protein Kinase C (e.g., PKCα). In some embodiments, contact or exposure of the peptide to a cell reduces or prevents redistribution or localization of eNOS to the mitochondria, reduces or prevents production of NOS-derived superoxide or mitochondrial reactive oxygen species (ROS), reduces or prevents loss of mitochondrial membrane potential, or combinations thereof. The contact or exposure of the peptide can be to a cell that is in a subject, such as a human.

Also disclosed are compositions including one or more of the peptides. For example, pharmaceutical compositions including an effective amount of the peptide or a plurality of copies of the peptide and a pharmaceutically acceptable carrier and/or diluent are provided.

Uses for the peptides and compositions thereof are provided for treating a disease disorder, or condition and/or reducing or preventing one or more symptoms of a disease disorder, or condition, in a subject in need thereof. Typically, the methods involve administering to the subject an effective amount of the pharmaceutical compositions.

The disease, disorder, or condition can be associated with disruption of the endothelial barrier (e.g., increased vascular permeability). Non-limiting diseases or disorders include pulmonary hypertension (PH), gram positive sepsis, acute lung injury (ALI), ventilator-induced lung injury (VILI), chronic obstructive pulmonary disease (COPD), acute respiratory distress syndrome (ARDS), pulmonary fibrosis, systemic inflammatory response syndrome (SIRS), multiorgan dysfunction syndrome (MODS), viral inflammation including COVID-19 induced ALI, and edema. In some embodiments, the disease is an inflammatory lung disease (e.g., PH, VILI, sepsis).

The composition is typically administered in an effective amount to treat, prevent or manage one of more of the symptoms of the disease. In some embodiments, the amount administered is effective to reduce or prevent inflammation or hypercytokinemia (cytokine storm) in the subject. In some embodiments, for example when the subject has ALI or ARDS, the amount of the composition administered is effective to reduce vascular leakage or permeability (e.g., in the lungs), to reduce bronchial alveolar lavage (BAL) protein levels, to reduce BAL cell count, to increase endothelial cell barrier integrity, reduce inflammation (e.g., in the lungs), or combinations thereof.

Administration of the compositions can be performed as necessary. Administration may occur in the intensive care setting. For example, the compositions can be administered prior to, during, or after mechanical ventilation of the subject. The compositions may be used preventatively prior to or when being put on mechanical ventilation in order to prevent or minimize lung injury associated with ventilator use. The compositions can be formulated for local or systemic delivery. In some embodiments, administration is by inhalation (e.g., of an aerosol), intratracheal instillation, or intravenous administration.

Preferably, the subject treated in accordance with any of the foregoing methods is human.

### BRIEF DESCRIPTION OF THE DRAWINGS

FIG. 1 shows that TRPV4 activation disrupts the endothelial barrier in pulmonary arterial endothelial cells. FIGS. 1A-1B are graphs showing cytosolic free Ca²⁺ levels on the y-axis in control (FIG. 1A) or 4αPDD-treated (right) PAECs. The TRPV4 agonist, 4αPDD (10 μM) induced a transient increase in cytosolic free Ca²⁺ concentration ([Ca²⁺]cyt) in PAEC (FIG. 1B). FIG. 1C is a graph showing normalized TER on the y-axis as a function of time (x-axis) upon treatment of cells with different doses of 4αPDD (0-15 μM). Data are mean±SEM. n=3. *P<0.05 vs. untreated.

FIGS. 2A-2F are a series of graphs showing that TRPV4 activation disrupts mitochondrial bioenergetics in pulmonary arterial endothelial cells. FIGS. 2A, 2B are graphs showing levels of mitochondrial ROS (FIG. 2A) and mitochondrial membrane potential (FIG. 2B) in vehicle or 4αPDD (10 μM, 3 h) treated PAECs. FIG. 2C is a graph showing changes in oxygen consumption rate (OCR) on the y-axis with vehicle or 4αPDD treatment. FIGS. 2D-2F are graphs showing levels of basal mitochondrial respiration, spare respiratory capacity, and maximal respiratory capacity upon vehicle or 4αPDD treatment. In FIGS. 2A and 2D, the left bar corresponds to vehicle treatment and the right bar corresponds to 4αPDD treatment. Values are mean±SEM; n=9-10. *P<0.05 vs. untreated.

FIGS. 3A-3I are graphs showing that TRPV4 activation induces the uncoupling and mitochondrial redistribution of eNOS in pulmonary arterial endothelial cells. FIG. 3A is a bar graph showing PKC levels on the y-axis in control (left most bar) and 4αPDD (10 μM) treated PAECs at 2, 4 and 6 hours. FIG. 3B is a bar graph showing eNOS T495 phosphorylation levels on the y-axis in control (left bar) and 4αPDD treated (right bar) cells. FIG. 3C is a bar graph showing NOS derived superoxide levels on the y-axis in control (left bar) and 4αPDD treated (right bar) cells. FIG. 3D is a bar graph showing cellular peroxynitrite levels on the y-axis in control (left bar) and 4αPDD treated (right bar) cells. FIG. 3E is a bar graph showing eNOS mitochondrial redistribution on the y-axis in control (left bar) and 4αPDD treated (right bar) cells. FIG. 3F is a bar graph showing pT495-eNOS levels on the y-axis in static (left bar) and cyclic stretch (18% stretch, 1 Hz, 4 h) (right bar) conditions. FIG. 3G is a bar graph showing eNOS mitochondrial redistribution on the y-axis in static (left bar) and cyclic stretch (right bar) conditions. FIG. 3H is a bar graph showing pT495-eNOS levels on the y-axis in static (left bar) and laminar shear stress (20 dyn/cm2, 4 h) (right bar) conditions. FIG. 3I is a bar graph showing eNOS mitochondrial redistribution on the y-axis in static (left bar) and laminar shear stress (right bar) conditions. Values are mean±SEM; n=3-10. *P<vs. untreated.

FIGS. 4A-4F are graphs showing that PKC activation disrupts mitochondrial bioenergetics in pulmonary arterial endothelial cells. FIG. 4A is a graph showing pT495 eNOS levels on the y-axis in control (left most bar) or PMA (100 nM) treated cells at 10, 30, and 60 minutes. FIG. 4B is a bar graph showing NOS derived superoxide levels on the y-axis in control (left bar) and PMA treated (right bar) cells. FIG. 4C is a bar graph showing changes in oxygen consumption rate (OCR) on the y-axis with control or PMA treatment. FIGS. 4D and 4E are bar graphs showing reserve- and maximal-respiratory capacities on the y-axis in control (left bar) and PMA treated (right bar) cells. FIG. 4F is a bar graph showing eNOS mitochondrial redistribution on the y-axis in control (left bar) and PMA treated (right bar) cells. Values are mean±SEM; n=3-10. *P<0.05 vs. untreated.

FIGS. 5A-5K are graphs showing that the over-expression of a constitutively active PKCα mutant mimics the effects of TRPV4 activation in pulmonary arterial endothelial cells. PAECs were transiently transfected with a constitutively active PKCα mutant (myr-PKCα) for 48 h. FIG. 5A is a bar graph showing PKCα levels on the y-axis in control (left bar) and myr-PKCα expressing PAECs (right bar). FIG. 5B is a bar graph showing pT495 eNOS levels on the y-axis in control (left bar) and myr-PKCα expressing cells (right bar). FIG. 5C is a bar graph showing NOS derived superoxide levels on the y-axis in control (left bar) and myr-PKCα expressing cells (right bar). FIG. 5D is a bar graph showing mitochondrial ROS levels on the y-axis in control (left bar) and myr-PKCα expressing cells (right bar). FIG. 5E is a bar graph showing mitochondrial membrane potential on the y-axis in control (left bar) and myr-PKCα expressing cells (right bar). FIG. 5F is a graph showing changes in oxygen consumption rate (OCR) on the y-axis in control and myr-PKCα expressing cells. Figures are a series of four bar graphs showing levels of basal O₂ consumption, ATP synthesis, reserve-, and maximal-respiratory capacity on the y-axis in control (left bar) and myr-PKCα expressing cells (right bar). FIG. 5K is a bar graph showing levels of eNOS mitochondrial colocalization on the y-axis in control (left bar) and myr-PKCα expressing cells (right bar). Values are mean±SEM; n=3-10. *P<0.05 vs. untreated.

FIGS. 6A-6G are graphs showing that blocking eNOS phosphorylation at T495 attenuates endothelial barrier disruption in vitro and in vivo. The T495 eNOS decoy peptide (d-peptide) is able to bind efficiently to purified PKCα, but not eNOS. FIG. 6A is a bar graph showing pT495 eNOS levels on the y-axis in response to treatment of PAECs with the d-peptide (1 μg/ml) and/or PMA (100 nm, 2 h). FIG. 6B is a bar graph showing NOS-derived superoxide levels on the y-axis in response to treatment of PAECs with the d-peptide and/or PMA. FIG. 6C is a bar graph showing eNOS mitochondrial colocalization levels on the y-axis in response to treatment of PAECs with the d-peptide and/or PMA. FIG. 6D is a graph showing change in normalized TER levels on the y-axis as a function of time (x-axis) in control, d-peptide plus 4αPDD, and 4αPDD treatment conditions. FIGS. 6E-6G show the effect of d-peptide in the mouse model of VILI. FIG. 6E is a bar graph showing pT495 eNOS levels on the y-axis in response to treatment with the d-peptide in a mouse model of VILI. FIG. 6F is a bar graph showing BALF cell number on the y-axis (as a measure of capillary permeability) in response to treatment with the d-peptide in a mouse model of VILI. FIG. 6G is a bar graph showing BALF protein concentration on the y-axis in response to treatment with the d-peptide in a mouse model of VILI. Values are mean±SEM; n=3-10. *P<0.05 vs. untreated. †P<0.05 vs PMA or VILI alone.

FIG. 7 is a schematic illustrating the mechanism underlying eNOS mediated mitochondrial dysfunction and oxidative stress. Specific activation of the mechanosensor, Ca²⁺-channel TRPV4, may lead to PKC-dependent phosphorylation of eNOS followed by its translocation to mitochondria and uncoupling. Translocated uncoupled eNOS induces mitoROS generation and oxidative/nitrosative stress that is characteristic for ARDS/VILI.

### DETAILED DESCRIPTION OF THE INVENTION

### I. Definitions

As used herein in reference to the peptides, the term “isolated” means a peptide that is in a form that is relatively free from material such as contaminating polypeptides, lipids, nucleic acids and other cellular material that normally is associated with the peptide in a cell or that is associated with the peptide in a library or in a crude preparation.

The term, “in vitro” refers to an artificial environment and to processes or reactions that occur within an artificial environment. In vitro environments include, but are not limited to, in solution or suspension, and cell cultures. The term “in vivo” refers to in or associated with an animal).

The terms “contact”, “contacting” or “exposing” refer to physical association. For example, to expose a peptide to a cell is to provide contact between the cell and the peptide. The term encompasses penetration of the contacted peptide to the interior of the cell by any suitable means, e.g., via transfection, electroporation, transduction, nanoparticle delivery, etc.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiologic effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

As used herein, “treatment” or “treating” means to administer a composition to a subject or a system with a condition to be treated, such as a disease or disorder. “Prevention” or “preventing” means to administer a composition to a subject or a system at risk for the condition. The condition can include a predisposition to a disease. The effect of administration of the composition to the subject (either treating and/or preventing) is to reduce duration or severity of, prevent or cease one or more symptoms of the condition.

As used herein, the terms “reduce” and “inhibit” mean to decrease an activity, response, condition, disease, or other biological parameter. This can include, but is not limited to, the complete ablation of the activity, response, condition, or disease. It is understood that this is typically in relation to a standard or expected value. The reduction or inhibition may be a 10, 20, 30, 40, 50, 60, 70, 80, 90, 100%, or any amount of reduction in between as compared to native or control levels. In some embodiments, inhibition or reduction is relative to a state prior to administration of one or more therapeutics. In some embodiments, inhibition or reduction is relative to a control that is not administered one or more therapeutics.

The term “binding” refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, peptides, nucleic acids, glycoproteins, carbohydrates, or endogenous small molecules. By “specific binding” or “selective binding” is meant that the molecules, such as peptides, that are able to bind to or recognize a binding partner (or a limited number of binding partners) to a substantially higher degree than to other, similar biological entities. For example, the molecule binds preferentially to the target as compared to non-target. Selective binding to is generally characterized by at least a two-fold greater binding to a target, as compared to a non-target. A molecule can be characterized by, for example, 5-fold, 10-fold, 20-fold or more preferential binding to the target as compared to one or more non-targets.

By “pharmaceutically acceptable” is meant a material that can be administered to a subject without causing undesirable biological effects or interacting in a deleterious manner with any of the other components of the pharmaceutical composition in which it is contained.

The term “percent (%) sequence identity” is defined as the percentage of nucleotides or amino acids in a candidate sequence that are identical with the nucleotides or amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or includes a certain % sequence identity to, with, or against a given sequence D) is calculated as follows:
100 times the fraction W/Z,
   where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5.

### II. Compositions

Disclosed are compositions containing isolated peptides that can serve as decoys for eNOS. The peptide(s) can be bound and/or phosphorylated by Protein Kinase C in place of eNOS, thus reducing or preventing phosphorylation dependent inhibition of eNOS.

A. Peptides

Disclosed are isolated, synthetic peptides capable of binding to Protein Kinase C (PKC), either in vitro or in vivo. In some embodiments, the peptide can be phosphorylated by Protein Kinase C (e.g., PKCα, PKGδ) in vitro or in vivo, such as at a threonine residue.

Because the peptide can serve as an endothelial nitric oxide synthase (eNOS) decoy, the peptide can effect reduced activity of one or more enzymes towards eNOS. For example, contact or exposure of the peptide to a cell can reduce or prevent phosphorylation of eNOS in the cell. In particular, the phosphorylation of endogenous eNOS at a threonine residue (e.g., T495) can be reduced or prevented. Thus, the peptide can reduce or prevent kinase activity of Protein Kinase C (e.g., PKCα) towards eNOS.

Since increased pT495-eNOS levels is associated with eNOS inactivation and uncoupling (F. Chen, PLoS One, 9(7), e99823 (2014); X. Sun, American Journal of Respiratory Cell and Molecular Biology, 50(6), 1084-95 (2014); S. Ghosh, Am J Physiol Lung Cell Mol Physiol, 310(11), L1199-205 (2016)), contact or exposure of the peptide to a cell can reduce or prevent eNOS inactivation and/or uncoupling. eNOS uncoupling refers to altered function of eNOS, such that it produces superoxide instead of nitric oxide (NO). Because uncoupled eNOS generates superoxide at the expense of NO, uncoupled eNOS contributes substantially to oxidative stress and endothelial dysfunction. Mechanisms of eNOS uncoupling include deficiency of the eNOS cofactor tetrahydrobiopterin, deficiency of the eNOS substrate L-arginine, and eNOS S-glutathionylation.

In some embodiments, contact or exposure of the peptide to a cell can reduce or prevent redistribution or localization of eNOS to the mitochondria (e.g., from the plasma membrane). In some embodiments, contact or exposure of the peptide to a cell can reduce levels of NOS-derived superoxide or mitochondrial reactive oxygen species (ROS). Contact or exposure of the peptide to a cell can reduce or prevent loss of mitochondrial membrane potential. In some embodiments, contact or exposure of the peptide to a cell can reduce or prevent disruption of mitochondrial bioenergetics, such as reductions in mitochondrial basal O₂ consumption, spare respiratory capacity and maximum respiratory capacity. For example, contact or exposure of the peptide to a cell can increase mitochondrial respiratory capacity.

In some embodiments, cellular peroxynitrite levels are reduced upon exposure or contact of a cell with the peptide(s). Peroxynitrite is thought to be generated by reaction of superoxide with nitric oxide.

In some embodiments, contact or exposure of the peptide to a cell can reduce or prevent activation of an inflammasome, such as the NLRP3 inflammasome. Thus, contact or exposure of the peptide to a cell can reduce or prevent inflammation (e.g., by reducing or preventing induction of one or more inflammatory cytokines).

In some embodiments, the peptides can cause any combination of the foregoing effects upon exposure or contact with a cell. The contact or exposure of the peptide can be to a cell that is in a subject, such as a human Thus, the disclosed effects can be achieved upon administration of a composition of the peptides to the subject. The compositions can include a plurality of copies of the peptide.

In some embodiments, the peptide includes the amino acid sequence HRKKRRQRRITRKKTFKEVA (SEQ ID NO:1). In some embodiments, the peptide includes an amino acid sequence having at least 70%, 75%, 80%, 85%, 90%, 95%, or more sequence identity to HRKKRRQRRITRKKTFKEVA (SEQ ID NO:1).

In some embodiments, the peptide is derived from and/or shows sequence similarity to the eNOS amino acid sequence or a portion thereof. Amino acid sequences of the human eNOS enzyme are known in the art. See, for example, UniProt ID No. P29474, which provides the following sequence:

(SEQ ID NO: 2)
MGNLKSVAQEPGPPCGLGLGLGLGLCGKQGPATPAPEPSRAPASLLPPA
PEHSPPSSPLTQPPEGPKFPRVKNWEVGSITYDTLSAQAQQDGPCTPRR
CLGSLVFPRKLQGRPSPGPPAPEQLLSQARDFINQYYSSIKRSGSQAHE
QRLQEVEAEVAATGTYQLRESELVFGAKQAWRNAPRCVGRIQWGKLQVF
DARDCRSAQEMFTYICNHIKYATNRGNLRSAITVFPQRCPGRGDFRIWN
SQLVRYAGYRQQDGSVRGDPANVEITELCIQHGWTPGNGRFDVLPLLLQ
APDDPPELFLLPPELVLEVPLEHPTLEWFAALGLRWYALPAVSNMLLEI
GGLEFPAAPFSGWYMSTEIGTRNLCDPHRYNILEDVAVCMDLDTRTTSS
LWKDKAAVEINVAVLHSYQLAKVTIVDHHAATASFMKHLENEQKARGGC
PADWAWIVPPISGSLTPVFHQEMVNYFLSPAFRYQPDPWKGSAAKGTGI
TRKK [symbol] FKEVANAVKISASLMGTVMAKRVKATILYGSETGRAQSYAQQL
GRLFRKAFDPRVLCMDEYDVVSLEHETLVLVVTSTEGNGDPPENGESFA
AALMEMSGPYNSSPRPEQHKSYKIRFNSISCSDPLVSSWRRKRKESSNT
DSAGALGTLRFCVFGLGSRAYPHFCAFARAVDTRLEELGGERLLQLGQG
DELCGQEEAFRGWAQAAFQAACETFCVGEDAKAAARDIFSPKRSWKRQR
YRLSAQAEGLQLLPGLIHVHRRKMFQATIRSVENLQSSKSTRATILVRL
DTGGQEGLQYQPGDHIGVCPPNRPGLVEALLSRVEDPPAPTEPVAVEQL
EKGSPGGPPPGWVRDPRLPPCTLRQALTFELDITSPPSPQLLRLLSTLA
EEPREQQELEALSQDPRRYEEWKWFRCPTLLEVLEQFPSVALPAPLLLT
QLPLLQPRYYSVSSAPSTHPGEIHLTVAVLAYRTQDGLGPLHYGVCSTW
LSQLKPGDPVPCFIRGAPSFRLPPDPSLPCILVGPGTGIAPFRGFWQER
LHDIESKGLQPTPMTLVFGCRCSQLDHLYRDEVONAQQRGVFGRVLTAF
SREPDNPKTYVQDILRTELAAEVHRVLCLERGHMFVCGDVTMATNVLQT
VQRILATEGDMELDEAGDVIGVLRDQQRYHEDIFGLTLRTQEVTSRIRT
QSFSLQERQLRGAVPWAFDPPGSDINSP.

Thus, the peptide can have for example, at least 70%, 75%, 80%, 85%, 90%, 95%, 99% or 100% amino acid sequence identity to SEQ ID NO:2 or a portion of SEQ ID NO:2. For example, protein kinase C-mediated phosphorylation of eNOS occurs at Thr495 (X. Sun, American Journal of Respiratory Cell and Molecular Biology, 50(6), 1084-95 (2014)), a threonine residue within the calmodulin-binding domain of eNOS (see, for example, residues 491-510 shown in double underline in SEQ ID NO:2 above). In some embodiments, the peptide can have for example, at least 70%, 75%, 80%, 85%, 90%, 95%, 99% or 100% amino acid sequence identity to the calmodulin-binding domain of eNOS (TRKKTFKEVANAVKISASLM; SEQ ID NO:3) or a portion thereof. In some embodiments, the peptide sequence includes, but is not limited to, an amino acid sequence having the residue at position 495 of an eNOS protein (e.g., T495; see highlighted residue in SEQ ID NO:2 above) and 1-5 amino acids immediately upstream and downstream of position 495 in the eNOS protein. Thus, in some embodiments, the peptide includes, but is not limited to, the amino acid sequence ITRKKTFKEVA (SEQ ID NO:4), wherein the underlined residue indicates T495 of the eNOS amino acid sequence of SEQ ID NO:2.

In some embodiments, the peptide sequence includes an amino acid sequence having a Protein Kinase consensus binding sequence. For example, the peptide can include a Protein Kinase C consensus binding sequence. An exemplary Protein Kinase C consensus binding sequence is X-S/T-X-R/K (SEQ ID NO:5), wherein X is any amino acid. Thus, in some embodiments, a suitable peptide is or minimally includes KTFK (SEQ ID NO:6). For example, suitable peptides can include a Protein Kinase consensus binding sequence in addition to 1-26 other amino acids, such as TRKKTFKEVA (SEQ ID NO:7), wherein the underlined residue indicates a PKC consensus binding sequence.

In other embodiments, suitable peptides include the combination of a Protein Kinase consensus binding and a functional peptide, such as a cell penetrating peptide (CPP). Due to their membrane penetrating ability, inclusion of a CPP in the peptide is expected to increase its cell penetration. The CPP can be positioned N- or C-terminal to the Protein Kinase consensus binding sequence. CPPs are known in the art and include, for example, CPPs described by Xie J., et al., Front Pharmacol., 11:697 (2020), which is hereby incorporated by reference in its entirety. Exemplary CPPs that can be incorporated into the disclosed PKC binding peptides include, but are not limited to, cationic, amphipathic and hydrophobic CPPs listed in Table 1.

TABLE 1
Exemplary Cell Penetrating Peptides

| | Peptide | Sequence |
|---|---|---|
| Cationic CPPs | TAT | RKKRRQRRR (SEQ ID NO: 8) |
| | R8 | RRRRRRRR (SEQ ID NO: 9) |
| | DPV3 | RKKRRRESRKKRRRES (SEQ ID NO: 10) |
| | DPV6 | GRPRESGKKRKRKRLKP (SEQ ID NO: 11) |
| | Penetratin | RQIKIWFQNRRMKWKK (SEQ ID NO: 12) |
| | R9-TAT | GRRRRRRRRRPPQ (SEQ ID NO: 13) |
| Amphipathic | pVEC | LLIILRRRIRKQAHAHSK (SEQ ID NO: 14) |
| CPPs | ARF (19-31) | RVRVFVVHIPRLT (SEQ ID NO: 15) |
| | MPG | GALFLGFLGAAGSTMGAWSQPKKKRKV |
| | | (SEQ ID NO: 16) |
| | MAP | KLALKLALKALKAALKLA (SEQ ID |
| | | NO: 17) |
| | Transportan | GWTLNSAGYLLGKINLKALAALAKKIL |
| | | (SEQ ID NO: 18) |
| Hydrophobic | Bip4 | VSALK (SEQ ID NO: 19) |
| CPPs | C105Y | CSIPPEVKFNPFVYLI (SEQ ID NO: 20) |
| | Melittin | GIGAVLKVLTTGLPALISWIKRKRQQ |
| | | (SEQ ID NO: 21) |
| | gH625 | HGLASTLTRWAHYNALIRAF (SEQ ID |
| | | NO: 22) |

Thus, an exemplary peptide suitable for use in accordance with the disclosed compositions and methods is the peptide having the amino acid sequence

(SEQ ID NO: 1)
HRKKRRQRRITRK [symbol] EVA,

wherein a TAT CPP sequence is underlined and a PKC consensus binding sequence is shown in bold. The portion of the sequence derived from the human eNOS protein is shown in italicized font.

The peptide can be of any length or size, as long as it retains functionality (e.g., binding to PKC, reducing or preventing eNOS phosphorylation and/or uncoupling). In some embodiments, the peptide can have a length of up to 30 residues. For example, the peptide can have a length of about 4-30 residues, such as about 4-20 residues or about 10-15 residues. In particular embodiments, the peptide has a length of 9, 10, 11, 12, 13, 14, 15, or 20 residues.

Suitable peptides also include variants of the peptides, such as the peptides of SEQ ID NOs:1-7, and modifications thereof retaining the same binding specificity. For example, suitable peptides can include one or more point mutations or substitutions (e.g., 1, 2, 3, 4, 5 or more mutations) at any amino acid residue of any one of SEQ ID NOs:1-7, such as HRKKRRQRRITRKKTFKEVA (SEQ ID NO:1). The one or more substitutions can be conservative or non-conservative. For example, peptide HRKKRRQRRITRKKTFKEVA (SEQ ID NO:1) can be modified by substituting one or more of the non-polar amino acid residues (V, A), with another, similarly non-polar residue, such as I, G, or L. Alanine scanning of peptides is useful for identifying amino acids that can be modified without reducing binding or other properties of the peptide.

The term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical, but in all cases retain the same binding specificity, and therefore mechanism of action. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, and size. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of the peptides can include variants having about 50%, 60%, 70%, 80%, 90%, 95%, 96%, 97%, 98%, 99%, or more sequence identity to the peptide of interest. The term “conservative amino acid substitution”, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

B. Peptide Modifications

The peptides may be modified in various ways. In some embodiments, the modification(s) may render the peptides more stable (e.g., resistant to degradation in vivo) or more capable of penetrating into cells, or other desirable characteristic as will be appreciated by one skilled in the art. Such modifications include, without limitation, chemical modification, N terminus modification, C terminus modification, peptide bond modification, backbone modifications, residue modification, D-amino acids, or non-natural amino acids or others. An individual peptide may contain one or more modifications. In preferred embodiments, the peptides are stabilized against proteolysis. For example, the stability and activity of peptides can be improved by protecting some of the peptide bonds with N-methylation or C-methylation. It is believed that modifications, such as amidation, also enhance the stability of peptides to peptidases.

Modifications to the peptides generally should leave them functional. A peptide with a structural difference from naturally occurring forms of peptides can be considered a modified peptide. It is understood that there are numerous amino acid and peptide analogs which can be incorporated into the peptides. For example, there are numerous D amino acids or other non-natural amino acids which can be used. The opposite stereoisomers of naturally occurring peptides are disclosed, as well as the stereo isomers of peptide analogs. These amino acids can readily be incorporated into polypeptide chains by chemical synthesis or by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991); Zoller, Current Opinion in Biotechnology, 3:348-354 (1992); Ibba, Biotechnology & Genetic Engineering Reviews 13:197-216 (1995)), all of which are herein incorporated by reference at least for material related to amino acid analogs) Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

The peptides may contain naturally occurring α-amino acid residues, non-naturally occurring α-amino acid residues, and combinations thereof. The D-enantiomer (“D-α-amino acid”) of residues may also be used Amino acids useful for inclusion in the peptides include, but are not limited to, artificial amino acids. Incorporation of artificial amino acids such as beta or gamma amino acids and those containing non-natural side chains, and/or other similar monomers such as hydroxyacids are also contemplated, with the effect that the corresponding component is peptide-like in this respect.

Non-naturally occurring amino acids are not found or have not been found in nature, but they can by synthesized and incorporated into a peptide chain. Non-natural amino acids are known to those skilled in the art of chemical synthesis and peptide chemistry. Non-limiting examples of suitable non-natural amino acids (in L- or D-configuration) are azidoalanine, azidohomoalanine, 2-amino-5-hexynoic acid, norleucine, azidonorleucine, L-α-aminobutyric acid, 3-(1-naphthyl)-alanine, 3-(2-naphthyl)-alanine, p-ethynyl-phenylalanine, m-ethynyl-phenylalanine, p-ethynyl-phenylalanine, p-bromophenylalanine, p-idiophenylalanine, p-azidophenylalanine, and 3-(6-chloroindolyl) alanin.

In some embodiments, peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), CC-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the normal side chain, naturally presented on the carbon atom. These modifications can occur at any of the bonds along the peptide chain and even at several (e.g., 2, 3, 4 or more) at the same time.

The peptides can be utilized in a linear form, although it will be appreciated that in cases where cyclization does not severely interfere with peptide characteristics, cyclic forms of the peptides can also be used. In some embodiments, a peptide may have a non-peptide macromolecular group covalently attached to its amino and/or carboxy terminus. Non-limiting examples of such macromolecular groups are proteins, lipid-fatty acid, polyethylene glycol, and carbohydrates.

Peptidomimetics may optionally be used to inhibit degradation of the peptides by enzymatic or other degradative processes. The peptidomimetics can be produced by organic synthetic techniques. Non-limiting examples of suitable peptidomimetics include D amino acids of the corresponding L amino acids. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides as long as activity is preserved.

In some embodiments, the peptides can contain one or more of the following modifications: glycosylation, amidation, acetylation, acylation, alkylation, alkenylation, alkynylation, phosphorylation, sulphorization, hydroxylation, hydrogenation, cyclization, ADP-ribosylation, anchor formation, covalent attachment of a lipid or lipid derivative, methylation, myristylation, pegylation, prenylation, esterification, biotinylation, coupling of farnesyl or ubiquitination, a linker which allows for conjugation or functionalization of the peptide, or a combination thereof.

Either or both ends of a given linear peptide can be modified. For example, the peptides can be acetylated and/or amidated.

In some embodiments, when the peptide is a linear molecule, it is possible to place various functional groups at various points on the linear molecule which are susceptible to or suitable for chemical modification. In some embodiments, the functional groups improve the activity of the peptide with regard to one or more characteristics, including but not limited to, stability, penetration (e.g., through cellular membranes and/or tissue barriers), tissue localization, efficacy, decreased clearance, decreased toxicity, improved selectivity, improved resistance to expulsion by cellular pumps, and the like. Non-limiting examples of suitable functional groups are described in Green and Wuts, “Protecting Groups in Organic Synthesis”, the teachings of which are incorporated herein by reference.

In some embodiments, the peptides can be cyclized. As used herein in reference to a peptide, the term “cyclic” means a structure including an intramolecular bond between two non-adjacent amino acids or amino acid analogues. The cyclization can be effected through a covalent or non-covalent bond. Intramolecular bonds include, but are not limited to, backbone to backbone, side-chain to backbone and side-chain to side-chain bonds. A preferred method of cyclization is through formation of a disulfide bond between the side-chains of non-adjacent amino acids or amino acid analogs. Residues capable of forming a disulfide bond include, for example, cysteine (Cys), penicillamine (Pen), β,β-pentamethylene cysteine (Pmc), ββ-pentamethylene-β-mercaptopropionic acid (Pmp) and functional equivalents thereof. Cysteine residues can be used to cyclize or attach two or more peptides together. This can be beneficial to constrain peptides into particular conformations. See Rizo and Gierasch Ann. Rev. Biochem. 61:387 (1992), incorporated herein by reference.

A peptide also can cyclize, for example, via a lactam bond, which can utilize a side-chain group of one amino acid or analog thereof to form a covalent attachment to the N-terminal amine of the amino-terminal residue. Residues capable of forming a lactam bond include aspartic acid (Asp), glutamic acid (Glu), lysine (Lys), ornithine (orn), α,β-diamino-propionic acid, γ-amino-adipic acid (Adp) and M-(aminomethyl)benzoic acid (Mamb). Cyclization additionally can be effected, for example, through the formation of a lysinonorleucine bond between lysine (Lys) and leucine (Leu) residues or a dityrosine bond between two tyrosine (Tyr) residues. The skilled person understands that these and other bonds can be included in a cyclic peptide.

In some embodiments, the peptides can be modified to include one or more albumin-binding molecules or moieties. Such albumin-binding molecules or moieties can provide altered pharmacodynamics of the peptide, such as alteration of tissue uptake, penetration, or diffusion; enhanced efficacy; and increased half-life. For example, the serum half-life of a peptide can be increased by linking it to a serum albumin-binding moiety and administering the peptide to a subject. The resulting conjugate will associate with circulating serum albumin and will remain in the serum longer than if the peptide were administered in the absence of a serum albumin-binding moiety. Thus, in particular forms, albumin-binding molecules or moieties are used to increase the half-life and overall stability of a peptide that is administered to or enters the circulatory system of a subject. The albumin-binding moiety can be covalently or non-covalently linked, coupled or associated to the peptide at a site that keeps the albumin-binding site of the moiety intact and still capable of binding to a serum albumin, without compromising the desired prophylactic or therapeutic activity of the peptide. Exemplary albumin-binding molecules or moieties that can be used include, without limitation, fatty acids and derivatives thereof, small molecules, peptides, and proteins. See Zorzi A., et al., MedChemComm., 10(7):1068-1081 (2019), which is hereby incorporated by reference in its entirety, and which provides a review of albumin-binding ligands and their use in extending the circulating half-life of therapeutics.

C. Pharmaceutical Compositions

The peptides are typically administered to a subject in need thereof in a pharmaceutical composition. Such pharmaceutical compositions may contain the peptide(s) in combination with a pharmaceutically acceptable carrier or diluent. Suitable carriers, diluents and their formulations are described in Remington: The Science and Practice of Pharmacy (19th ed.) ed. A. R. Gennaro, Mack Publishing Company, Easton, PA 1995. Examples of pharmaceutically-acceptable carriers include, but are not limited to, saline, Ringer's solution and dextrose solution. It will be apparent to those persons skilled in the art that certain carriers can be more preferable depending upon, for instance, the route of administration and concentration of composition being administered. Those of skill in the art can readily determine the various parameters for preparing and formulating the compositions without resort to undue experimentation.

Pharmaceutical compositions can be manufactured by processes well known in the art, e.g., by means of conventional mixing, dissolving, granulating, dragee-making, levigating, emulsifying, encapsulating, entrapping or lyophilizing processes.

Pharmaceutical compositions may be formulated in a conventional manner using one or more physiologically acceptable carriers including excipients and auxiliaries which facilitate processing of the active compounds into preparations which can be used pharmaceutically. The compositions may be administered in combination with one or more physiologically or pharmaceutically acceptable carriers, thickening agents, co-solvents, adhesives, antioxidants, buffers, viscosity and absorption enhancing agents and agents capable of adjusting osmolarity of the formulation. Proper formulation is dependent upon the route of administration chosen. If desired, the compositions may also contain minor amounts of nontoxic auxiliary substances such as wetting or emulsifying agents, dyes, pH buffering agents, or preservatives.

Pharmaceutical compositions of the peptides may be for systemic or local administration. In some embodiments, the compositions can be formulated for administration by parenteral (e.g., intramuscular (IM), intraperitoneal (IP), intravenous (IV), intra-arterial, intrathecal or subcutaneous injection (SC)), or transmucosal (nasal, vaginal, pulmonary, or rectal) routes of administration.

The compositions may be formulated for parenteral administration by injection, e.g., by bolus injection or continuous infusion. For injection, the peptides and compositions thereof may be formulated in aqueous solutions, preferably in physiologically compatible buffers such as Hanks's solution, Ringer's solution, or physiological saline buffer. Formulations for injection may be presented in unit dosage form, e.g., in ampules or in multi-dose containers, optionally with an added preservative. The compositions may take such forms as sterile aqueous or non-aqueous solutions, suspensions and emulsions, which can be isotonic with the blood of the subject in certain embodiments. Examples of non-aqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). The compositions may be in solution, emulsions, or suspension (for example, incorporated into particles or liposomes). Typically, an appropriate amount of a pharmaceutically-acceptable salt is used in the formulation to render the formulation isotonic. Trehalose, typically in the amount of 1-5%, may be added to the pharmaceutical compositions. The pH of the solution is preferably from about 5 to about 8, and more preferably from about 7 to about 7.5.

Enteral administration (e.g., oral, sublingual) may be used where the peptides are stable enough to withstand the harsh proteolytic environment of the gastrointestinal tract. If so, the compositions can be formulated readily by combining the peptide compositions with pharmaceutically acceptable carriers well known in the art. Such carriers enable the compositions to be formulated as tablets, pills, dragees, capsules, liquids, gels, syrups, slurries, suspensions and the like, for oral ingestion by a patient to be treated. Pharmacological preparations for oral use can made with the use of a solid excipient, optionally grinding the resulting mixture, and processing the mixture of granules, after adding suitable auxiliaries, if desired, to obtain tablets. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Formulations for Mucosal and Pulmonary Administration

In some embodiments, the compositions are formulated for pulmonary or mucosal administration, such as through nasal, pulmonary, oral (e.g., sublingual, buccal), vaginal, or rectal mucosa delivery.

Preferably, the peptides are formulated into pharmaceutical compositions for pulmonary delivery, such as intranasal administration or inhalation. Carriers for pulmonary formulations can be divided into those for dry powder formulations and for administration as solutions. In some embodiments, the pharmaceutical compositions can be inhalable or aerosolized. Suitable pharmaceutical compositions for inhaled administration will typically be in the form of an aerosol or a powder. Such compositions are generally administered using well-known delivery devices, such as a nebulizer inhaler, a metered-dose inhaler (MDI), a dry powder inhaler (DPI) or a similar delivery device.

Intranasal compositions may be administered using devices known in the art, for example a nebulizer or nasal sprayer or injector. For example, a device for intranasal administration can be loaded with the pharmaceutical composition of the peptides. The device can include a sprayer equipped with a nozzle that upon intranasal administration produces a fine mist of the pharmaceutical composition that is primarily deposited in the subject's nose and nasopharynx. For administration by inhalation, the pharmaceutical composition can be delivered in the form of an aerosol spray presentation from pressurized packs or a nebulizer, with the use of a suitable propellant, e.g., dichlorodifluoromethane, trichlorofluoromethane, dichloro-tetrafluoroethane, carbon dioxide, propane, nitrogen, air or other suitable gas. The term aerosol refers to any preparation of a fine mist of particles, which can be in solution or a suspension, whether or not it is produced using a propellant. Aerosols can be produced using standard techniques, such as ultrasonication or high-pressure treatment. In the case of a pressurized aerosol, the dosage unit may be determined by providing a valve to deliver a metered amount. Aerosols for the delivery of therapeutics to the respiratory tract are known in the art.

Inhalable as used herein includes the state of being deliverable to the airway (e.g., respiratory tract, lungs). The inhaled route allows the delivery of a therapeutic directly to the airway achieving a high local concentration while minimizing systemic delivery and/or adverse effects. As a result, considerably lower inhaled doses can be therapeutically equivalent or even superior to higher doses of systemically administered therapy. An inhalable form or formulation may contain a powder, liquid particles, or solid particles of the therapeutic of a size sufficiently small to pass through the mouth and larynx upon inhalation and continue into the bronchi and alveoli of the lungs.

For administration via the upper respiratory tract, the pharmaceutical composition can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

Mucosal formulations may include one or more agents for enhancing delivery through the nasal mucosa. Agents for enhancing mucosal delivery are known in the art, see, for example, U.S. Patent Application No. 2009/0252672 to Eddington, and U.S. Patent Application No. 2009/0047234 to Touitou. Acceptable agents include, but are not limited to, chelators of calcium (EDTA), inhibitors of nasal enzymes (boro-leucin, aprotinin), inhibitors of muco-ciliar clearance (preservatives), solubilizers of nasal membrane (cyclodextrin, fatty acids, surfactants) and formation of micelles (surfactants such as bile acids, Laureth 9 and taurodehydrofusidate (STDHF)). Compositions may include one or more absorption enhancers, including surfactants, fatty acids, and chitosan derivatives, which can enhance delivery by modulation of the tight junctions (TJ) (B. J. Aungst, et al., J. Pharm. Sci. 89(4):429-442 (2000)). In general, the optimal absorption enhancer should possess the following qualities: its effect should be reversible, it should provide a rapid permeation enhancing effect on the cellular membrane of the mucosa, and it should be non-cytotoxic at the effective concentration level and without deleterious and/or irreversible effects on the cellular or virus membrane.

### III. Methods of Use

Also provided are uses for the peptides and compositions thereof. The peptides and compositions thereof can be used in diagnostic, therapeutic and/or prophylactic applications.

Methods of Treatment

It has been discovered that phosphorylation-induced endothelial nitric oxide synthase (eNOS) inactivation and uncoupling contributes to the compromised endothelial cell barrier integrity that is often characteristic of VILI and other vascular permeability related disorders. As shown in the working examples, the peptides and compositions thereof can be used to block phosphorylation-induced eNOS inactivation and attenuate the resulting loss of barrier integrity. Thus, the peptide compositions provide a means to treat several diseases or disorders associated with increased vascular permeability.

Methods of using the disclosed eNOS decoy peptides including, but not limited to, methods designed to inhibit or block eNOS phosphorylation, mitochondrial localization and/or uncoupling in vivo, and methods to improve mitochondrial bioenergetics in vivo can be used to modulate cellular functions and prevent, reduce or reverse undesirable reduction in vascular permeability.

In preferred embodiments, the peptide compositions can be used to treat, prevent or manage a disease or condition, in a subject. The peptide compositions can also be used to reduce, manage, delay or prevent one or more symptoms of a disease disorder, or condition, in a subject in need thereof. In preferred embodiments, the subject is a human.

i. Diseases

The peptide compositions are useful for the treatment and/or prevention of diseases, disorders or conditions caused by abnormal eNOS inactivation or uncoupling. In particular, the peptide compositions are useful for the treatment and/or prevention of diseases, disorders or conditions associated with local or systemic disruption of endothelial barrier function (e.g., increased vascular permeability). Such diseases, disorders, and conditions include inflammatory diseases, particularly those associated with loss of or compromised endothelial barrier function (e.g., inflammatory lung disease). For example, the peptides may reduce lung inflammation, which correlates with reduced lung damage and lung edema. The peptide compositions are useful for the treatment and/or prevention of diseases, disorders or conditions caused by or associated with local or systemic inflammation, cytokine storm, and/or activation of inflammasomes.

Non-limiting examples of diseases, disorders or conditions include pulmonary hypertension (PH), sepsis (e.g., gram positive sepsis), acute lung injury (ALI), ventilator-induced lung injury (VILI), chronic obstructive pulmonary disease (COPD), acute respiratory distress syndrome (ARDS), pulmonary fibrosis, systemic inflammatory response syndrome (SIRS), multiorgan dysfunction syndrome (MODS), COVID-19, and edema (e.g., pulmonary edema). In some embodiments, the disease is an inflammatory lung disease (e.g., PH, VILI, sepsis).

Pulmonary Hypertension (PH)

Pulmonary hypertension (PH) is an elevation in the pressure in the arteries of the lungs. The clinical classification system for PH includes five distinct “Groups” (J Am Coll Cardiol, 54:S1-117 (2009); J Am Coll Cardiol, 54:S43-54 (2009)) including a very broad spectrum of disease etiology and pathobiology affecting not only the lungs and right ventricle directly, but also secondarily through other organ pathologies. Group I is pulmonary artery hypertension (PAH). Group II is PH associated with left heart disease. Group III is PH associated with lung disease and/or hypoxia. Group IV is PH associated with chronic thromboembolic disease, and Group V is PH associated with multifactorial mechanisms. Within each classification groups I-IV, there are distinct mechanistic programs that contribute to PH, either on the arterial or venous side of the pulmonary circulation. The methods can treat pulmonary hypertension (PH) classified into any one of the five clinically-recognized groups.

Pulmonary arterial hypertension (PAH) is a specific subgroup of pulmonary hypertension (PH), characterized by high blood pressure (hypertension) of the main artery of the lungs (pulmonary artery) for no apparent reason (idiopathic). PAH is a rare, progressive disorder with an estimated prevalence of 15 to 50 cases per 1 million people, usually affecting women between the ages of 20-50. Pulmonary arterial hypertension (PAH) is a currently fatal condition in which pulmonary vascular inflammation and remodeling leads to elevated pulmonary arterial pressure, right ventricular (RV) hypertrophy (RVH), and, ultimately, RV dysfunction and failure.

Clinical signs of PAH indicative of a need for treatment include any one or more of dyspnea, fatigue, and chest pain. Any of these symptoms can be present only with exertion, or both with exertion and at rest. Additional symptoms include syncope, edema and swelling, dizziness, poor or reduced oral intake, as well as any of the signs and symptoms of right heart failure, increased or faster than normal heart rate and palpitations.

Ventilator-Induced Lung Injury (VILI)

It is established that mechanical ventilation can injure the lung, producing an entity known as ventilator-induced lung injury (VILI). There are various forms of VILI, including volutrauma (i.e., injury caused by overdistending the lung), atelectrauma (injury due to repeated opening/closing of lung units), and biotrauma (release of mediators that can induce lung injury or aggravate pre-existing injury, potentially leading to multiple organ failure).

VILI can occur as a result of cyclic stretching and overdistension of the lung tissues, which cause severe inflammation and structural tissue damage ultimately leading to acute lung injury (ALI). Additional factors that contribute to VILI are the disease or events that led to respiratory failure, and the parameters used in mechanical ventilation treatment (volume, pressure, and duration). There are no efficient pharmacological strategies to ameliorate the negative effects caused by mechanical ventilation, and only a conservative approach using a low tidal volume has been shown to cause less damage.

VILI is characterized by a disruption of the alveolar-capillary barrier which increases permeability, thus causing edema, inflammatory leukocyte infiltration (mainly neutrophils), and hemorrhage. Stretch forces cause the release of inflammatory cytokines like IL6, IL8, IL1p, and TNFα by activation of the p38 MAPK pathway and of the transcription facto,r NF-κB Cyclic stretch also generates reactive oxygen species (ROS) that further exacerbate VILI. These events are followed by the onset of an endogenous anti-inflammatory and anti-oxidative reaction to compensate for and attenuate VILI-derived inflammatory response and redox imbalance.

Acute Respiratory Distress Syndrome (ARDS)

ARDS is defined as an acute condition characterized by bilateral pulmonary infiltrates and severe hypoxemia in the absence of evidence for cardiogenic pulmonary edema. Acute respiratory distress syndrome (ARDS) is associated with diffuse alveolar damage (DAD) and lung capillary endothelial injury. The early phase is described as being exudative, whereas the later phase is fibroproliferative in character. Early ARDS is characterized by an increase in the permeability of the alveolar-capillary barrier leading to an influx of fluid into the alveoli. The alveolar-capillary barrier is formed by the microvascular endothelium and the epithelial lining of the alveoli. Hence, a variety of insults resulting in damage either to the vascular endothelium or to the alveolar epithelium could result in ARDS. The main site of injury may be focused on either the vascular endothelium (e.g., sepsis) or the alveolar epithelium (e.g., aspiration of gastric contents).

Injury to the endothelium results in increased capillary permeability and the influx of protein-rich fluid into the alveolar space. Injury to the alveolar lining cells also promotes pulmonary edema formation. Two types of alveolar epithelial cells exist. Type I cells, comprising 90% of the alveolar epithelium, are injured easily. Damage to type I cells allows both increased entry of fluid into the alveoli and decreased clearance of fluid from the alveolar space. Type II cells have several important functions, including the production of surfactant, ion transport, and proliferation and differentiation into type I cells after cellular injury. Damage to type II cells results in decreased production of surfactant with resultant decreased compliance and alveolar collapse. Interference with the normal repair processes in the lung may lead to the development of fibrosis.

ARDS causes marked increase in intrapulmonary shunt, leading to severe hypoxemia. Although high inspired oxygen concentrations are required to maintain adequate tissue oxygenation and life, additional measures, like lung recruitment with positive end-expiratory pressure (PEEP), is often required. ARDS is uniformly associated with pulmonary hypertension. Pulmonary artery vasoconstriction likely contributes to ventilation-perfusion mismatch and is one of the mechanisms of hypoxemia in ARDS. Normalization of pulmonary artery pressures occurs as the syndrome resolves. Morbidity is considerable. Patients with ARDS are likely to have prolonged hospital courses, and they frequently develop nosocomial infections, especially ventilator-associated pneumonia. In addition, patients often have significant weight loss and muscle weakness and functional impairment may persist for months following hospital discharge. Most of the deaths in ARDS are attributable to sepsis, multiorgan failure and even lung injury through mechanical ventilation.

Acute Lung Injury (ALI)

Acute lung injury (ALI) is a diffuse heterogeneous lung injury characterized by hypoxemia, non-cardiogenic pulmonary edema, low lung compliance and widespread capillary leakage. ALI can be caused by any stimulus of local or systemic inflammation, principally sepsis.

Types of ALI include, primary ALI, which can be caused by a direct injury to the lung (e.g., pneumonia), and secondary ALI, which can be caused by an indirect insult (e.g., pancreatitis). There are two stages—the acute phase characterized by disruption of the alveolar-capillary interface, leakage of protein rich fluid into the interstitium and alveolar space, and extensive release of cytokines and migration of neutrophils. A later reparative phase is characterized by fibroproliferation and remodeling of lung tissue.

The core pathology is disruption of the capillary-endothelial interface: this actually refers to two separate barriers—the endothelium and the basement membrane of the alveolus. In the acute phase of ALI, there is increased permeability of this barrier, and protein rich fluid leaks out of the capillaries. There are two types of alveolar epithelial cells Type 1 pneumocytes represent 90% of the cell surface area, and are easily damaged. Type 2 pneumocytes are more resistant to damage, which is important as these cells produce surfactant, transport ions and proliferate and differentiate into Type 1 cells. The damage to the endothelium and the alveolar epithelium results in the creation of an open interface between the lung and the blood, facilitating the spread of micro-organisms from the lung systemically, stoking up a systemic inflammatory response. Moreover, the injury to epithelial cells handicaps the lung's ability to pump fluid out of airspaces. Fluid filled airspaces, loss of surfactant, microvascular thrombosis and disorganized repair (which leads to fibrosis) reduces resting lung volumes (decreased compliance), increasing ventilation-perfusion mismatch, right to left shunt and the work of breathing. In addition, lymphatic drainage of lung units appears to be curtailed—stunned by the acute injury: this contributes to the build-up of extravascular fluid. The ALI patient has low lung volumes, atelectasis, loss of compliance, ventilation-perfusion mismatch (increased deadspace), and right to left shunt. Clinical features are—severe dyspnea, tachypnea, and resistant hypoxemia.

Prolonged inflammation and destruction of pneumocytes leads to fibroblastic proliferation, hyaline membrane formation and lung fibrosis. This fibrosing alvcolitis may become apparent as early as five days after the initial injury. Subsequent recovery may be characterized by reduced physiologic reserve, and increased susceptibility to further lung injuries. Extensive microvascular thrombosis may lead to pulmonary hypertension, myocardial dysfunction and systemic hypotension.

Chronic Obstructive Pulmonary Disease (COPD)

Chronic obstructive pulmonary disease (COPD), also known as chronic obstructive lung disease (COLD), chronic obstructive airway disease (COAD), chronic airflow limitation (CAL) and chronic obstructive respiratory disease (CORD), refers to chronic bronchitis and emphysema, a pair of commonly co-existing diseases of the lungs in which the airways become narrowed. This leads to a limitation of the flow of air to and from the lungs causing shortness of breath. In clinical practice, COPD is defined by its characteristically low airflow on lung function tests. In contrast to asthma, this limitation is poorly reversible and usually gets progressively worse over time.

COPD is caused by noxious particles or gas, most commonly from tobacco smoking, which triggers an abnormal inflammatory response in the lung. The inflammatory response in the larger airways is known as chronic bronchitis, which is diagnosed clinically when people regularly cough up sputum. In the alveoli, the inflammatory response causes destruction of the tissues of the lung, a process known as emphysema. The natural course of COPD is characterized by occasional sudden worsening of symptoms called acute exacerbations, most of which are caused by infections or air pollution.

Both emphysematous destruction and small airway inflammation often are found in combination in individual patients, leading to the spectrum that is known as COPD. When emphysema is moderate or severe, loss of elastic recoil, rather than bronchiolar disease, is the mechanism of airflow limitation. By contrast, when emphysema is mild, bronchiolar abnormalities are most responsible for the deficit in lung function. Although airflow obstruction in emphysema is often irreversible, bronchoconstriction due to inflammation accounts for a limited amount of reversibility.

Pathological changes in chronic obstructive pulmonary disease (COPD) occur in the large (central) airways, the small (peripheral) bronchioles, and the lung parenchyma. The increased number of activated polymorphonuclear leukocytes and macrophages release elastases in a manner that cannot be counteracted effectively by antiproteases, resulting in lung destruction. The primary offender has been human leukocyte elastase, with a possible synergistic role suggested for proteinase 3 and macrophage-derived matrix proteinases, cysteine proteinases, and a plasminogen activator. Additionally, increased oxidative stress caused by free radicals in cigarette smoke, the oxidants released by phagocytes, and polymorphonuclear leukocytes all may lead to apoptosis or necrosis of exposed cells. Accelerated aging and autoimmune mechanisms have also been proposed as having roles in the pathogenesis of COPD.

Pulmonary Fibrosis (Idiopathic)

Idiopathic pulmonary fibrosis (IPF), the most fatal and progressive fibrotic lung disease, disproportionately affects the elderly population and is now widely regarded as a disease of aging. Idiopathic Pulmonary fibrosis (IPF) is a specific subgroup of pulmonary fibrosis. IPF is a lung disease that results in scarring (fibrosis) of the lungs for an unknown reason. Over time, the scarring gets worse and it becomes hard to take in a deep breath and the lungs cannot take in enough oxygen. IPF is a form of interstitial lung disease, primarily involving the interstitium (the tissue and space around the air sacs of the lungs), and not directly affecting the airways or blood vessels. The cause of idiopathic pulmonary fibrosis is not completely understood.

Aging and fibrotic disease are both associated with cumulative oxidant burden, and lung tissue from IPF patients demonstrate “signatures” of chronic oxidative damage. The lungs are particularly prone to insult and injury by oxygen free radicals given their direct exposure to the environment and inspired air.

Clinical signs of IPF indicative of a need for treatment include any one or more of dyspnea (i.e., breathlessness, shortness of breath), usually during exercise, chronic cough, chest pain or tightness, unexplained weight loss, loss of appetite, fatigue, and clubbing of the digits (i.e., change of finger shape). About 85% of people with IPF have a chronic cough that has lasts longer than 8 weeks. This is often a dry cough, but some people may also cough up sputum or phlegm. Breathlessness can affect day-to-day activities such as showering, climbing stairs, getting dressed and eating. As scarring in the lungs gets worse, breathlessness may prevent all activities.

ii. Effective Amounts

Typically, the methods involve administering to the subject an effective amount of the pharmaceutical compositions. For example, in some embodiments, the peptide compositions are administered to a subject in a therapeutically effective amount for treatment of one or more signs or symptoms of a disease, disorder or condition.

The effective amount or therapeutically effective amount can be a dosage sufficient to treat, inhibit, or alleviate one or more symptoms of the disorder, disorder or condition being treated or to otherwise provide a desired pharmacologic and/or physiologic effect. The effective amount of the peptide compositions will vary from subject to subject, depending on the species, age, weight and general condition of the subject, the severity of the disorder being treated, and its mode of administration. Thus, it is not possible to specify an exact amount for every therapeutic composition. However, an appropriate amount can be determined by one of ordinary skill in the art using only routine experimentation given the teachings herein. For example, effective dosages and schedules for administering the therapeutics may be determined empirically, and making such determinations is within the skill in the art. The dosage ranges for the administration of the compositions are those large enough to effect one or more desired responses.

As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage can depend upon the age, condition, and sex of the subject, the desired therapeutic effect, on the route of administration, and on the duration of the treatment desired. The dosage should not be so large as to cause adverse side effects, such as unwanted cross-reactions, anaphylactic reactions, and the like. The dosage can be adjusted by the individual physician in the event of any counter-indications. It will also be appreciated that the effective dosage of the composition used for treatment may increase or decrease over the course of a particular treatment. Changes in dosage may result and become apparent from the results of diagnostic assays.

In some embodiments, the amount of the peptide composition administered is effective to reduce or prevent inflammation or hypercytokinemia (cytokine storm) in the subject. In some embodiments, the amount of the composition administered is effective to reduce vascular leakage or permeability (e.g., in the lungs), to reduce bronchial alveolar lavage (BAL) protein levels, to reduce BAL cell count, to increase endothelial cell barrier integrity, reduce local or systemic inflammation (e.g., in the lung), reduce vascular permeability or leakage (e.g., in the lung), increase alveolar cell integrity, increase endothelial cell integrity, or combinations thereof. In some embodiments, the amount of the composition administered is effective to enhance pulmonary compliance in a subject.

In some embodiments, when the disease, disorder or condition is a lung injury, treatment of lung injury can be monitored by determining the level of PaO₂ using suitable techniques known in the art. Treatment can also be monitored by determining total and differential bronchoalveolar lavage (BAL) counts of different cell populations (e.g., neutrophils, lymphocytes, monocytes, eosinophils, basophils) using suitable techniques known in the art. Treatment can also be monitored by non-invasive scanning of the affected organ or tissue such as by computer assisted tomography scan, magnetic resonance elastography scans and other suitable techniques known in the art.

The peptide compositions can be administered to a subject at a suitable dose, such as from about 1 μg/kg to about 20 mg/kg, for example, from about 1 mg/kg to about 10 mg/kg.

An effective amount of the peptide composition can be compared to a control. Suitable controls are known in the art. A typical control is a comparison of a condition or symptom of a subject prior to and after administration of the composition. The condition or symptom can be a biochemical, molecular, physiological, or pathological readout. In another embodiment, the control is a matched subject that is administered a different agent or that does not receive any treatment. The compositions disclosed here can be compared to other art recognized treatments for the disease or condition to be treated or prevented.

iii. Timing of Administration and Dosage Regimens

Dosages and timing of administration can vary. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages may vary depending on the relative potency of individual therapeutics, and can generally be estimated based on EC₅₀s found to be effective in in vitro and in vivo animal models.

Treatment can be continued for an amount of time sufficient to achieve one or more desired goals (e.g., therapeutic or prophylactic goals). Treatment can be continued for a desired period of time, and the progression of treatment can be monitored using any suitable means known in the art. In some embodiments, administration is carried out every day of treatment, or every week, or every fraction of a week. In some embodiments, treatment regimens are carried out over the course of up to two, three, four or five days, weeks, or months, or for up to 6 months, or for more than 6 months, for example, up to one or two years.

The compositions can be administered during a period during, or after onset of disease symptoms, or any combination of periods during or after diagnosis of one or more disease symptoms. For example, the subject can be administered one or more doses of the composition every 1, 2, 3, 4, 5, 6, 7, 14, 21, 28, 35, or 48 days after the onset or diagnosis of disease symptoms. In some embodiments, the multiple doses of the compositions are administered before an improvement in disease condition is evident. For example, in some embodiments, the subject receives the composition, over a period of 1, 2, 3, 4, 5, 6 7, 14, 21, 28, 35, or 48 days or weeks before an improvement in the disease or condition is evident.

In some embodiments, the subject is a patient in intensive care. In the intensive care setting, the peptide compositions can be administered over the course of one or more hours, for example, as a rescue therapy or salvage therapy. In some embodiments, the peptide compositions can be administered as a preventative. For example, the composition can be administered to a subject prior to or at the time of mechanical ventilation in order to prevent or minimize lung injury associated with ventilator use. The composition can be administered hourly, daily, weekly, or monthly, one or more times, as required. In a particular embodiment, the compositions are delivered to the patient via intravenous infusion over the course of one or more hours.

iv. Routes of Administration

Any suitable route of administration can be used for the disclosed compositions. Routes of administration can, for example, include topical, enteral, local, systemic, or parenteral. In some embodiments, the compositions are administered via intravenous infusion (i.v.), intraperitoneally (i.p.), intramuscularly (i.m.), subcutaneously (s.c.), transdermally, topically, intranasally, or by endotracheal or intratracheal (i.t.) delivery. In some preferred embodiments, the peptide compositions are delivered to a subject by intravenous infusion. The compositions may be administered by injection, or by other means appropriate to a specific dosage form, e.g., including administration by inhalation of a lyophilized powder. In some embodiments, the pulmonary route of administration is preferred, such as by intratracheal instillation, inhalation (e.g., of an aerosol or powder formulation), although other routes, may be required in specific administration, as for example to the mucous membranes of the nose, throat, bronchial tissues or lungs.

### IV. Methods of Manufacture

In some embodiments, the peptides can be obtained commercially, such as from a vendor which provides custom peptide synthesis services.

In some embodiments, peptides having a desired sequence can be synthesized or produced recombinantly. Thus, methods of making the peptides using techniques known in the art are provided. Peptides are typically synthesized using standard procedures, so any technique in the art suitable to prepare synthetic peptides can be used. For example, standard FMOC synthesis is described in the literature (e.g., solid phase peptide synthesis, see E. Atherton, RC Sheppard, Oxford University press (1989), or liquid phase synthesis (where peptides are assembled using a mixed strategy by BOC chemistry and fragment condensation).

The peptides can be produced by recombinant means (e.g., in bacteria, yeast, fungi, insect, vertebrate or mammalian cells) by methods well known to those skilled in the art.

Alternatively, the peptides can be synthesized using techniques well-known to those skilled in the art, e.g., by standard solid-phase peptide synthesis. Such methods include bench scale solid phase synthesis and automated peptide synthesis in any one of the many commercially available peptide synthesizers. Solid phase synthesis is commonly used and various commercial synthesizers are available, for example automated synthesizers by Applied Biosystems Inc., Foster City, CA; Beckman; MultiSyntech, Bochum, Germany etc. Solution phase synthetic methods may also be used, although this can be less convenient. Functional groups for conjugating the peptide to small molecules, label moieties, peptides, or proteins may be introduced into the molecule during chemical synthesis. In addition, small molecules and label moieties/reporter units may be attached during the synthetic process. Preferably, introduction of the functional groups and conjugation to other molecules minimally affects the structure and function of the subject peptide.

The peptides can be produced by stepwise synthesis or by synthesis of a series of fragments that can be coupled by well-known techniques.

Chemical synthesis typically starts from the C-terminus, to which amino acids are sequentially added using either a Rink amide resin (resulting in an —NH₂ group at the C-terminus of the peptide after cleavage from the resin), or a Wang resin (resulting in an —OH group at the C-terminus). Accordingly, peptides having a C-terminal moiety that may be selected from the group consisting of —H, —OH, —COOH, —CONH₂, and —NH₂ are contemplated for use.

Standard Fmoc (9-florenylmethoxycarbonyl) derivatives include Fmoc-Asp(OtBu)-OH, Fmoc-Arg(Pbe-OH, and Fmoc-Ala-OH. Couplings are mediated with DIC (diisopropylcarbodiimide)/6-Cl-HOBT (6-chloro-1-hydroxybenzotriazole). In some embodiments, the last four residues of the peptide require one or more recoupling procedures. In particular, the final Fmoc-Arg(Pbf)-OH coupling can require recoupling. For example, a second or third recoupling can be carried out to complete the peptide using stronger activation chemistry such as DIC/HOAT (1-hydroxy-7-azabenzotriazole) or HATU (1-[bis(dimethylamino)methylene]-1H-1,2,3-triazolo[4,5-b]pyridinium 3-oxid hexafluorophosphate)/NMM (N-methylmorpholine).

Acidolytic cleavage of the peptide can be carried out with the use of carbocation scavengers (thioanisole, anisole and H₂O). Optimization can be achieved by varying the ratio of the components of the cleavage mixture. An exemplary cleavage mixture ratio is 90:2.5:2.5:5 (TFA-thioanisole-anisole-H₂O). The reaction can be carried out for 4 hours at room temperature.

In some embodiments, the removal of residual impurities is carried out by wash steps. For example, trifluoroacetic acid (TFA) and organic impurities can be eliminated by precipitation and repeated washes with cold diethyl ether and methyl t-butyl ether (MTBE).

Peptides produced using the disclosed methods can be purified using high pressure liquid chromatography (HPLC). Suitable solvents for dissolving the peptides include neat TFA. In some embodiments, 8 mL TFA/g peptide is sufficient to fully dissolve peptides following precipitation. For example, TFA can be diluted into H₂O. Typically, the peptides remain soluble at TFA concentrations of 0.5% to 8% and can be loaded onto reverse phase (RP)-HPLC columns for salt exchange. Exemplary salt exchange methods use 3-4 column volumes of acidic buffer to wash away the TFA counter ion due to its stronger acidity coefficient. Buffers suitable for use in washing away the TFA counter ion include 0.1% HCl in H₂O.

Following removal of TFA, peptides can be eluted with a step gradient. Exemplary elution buffers include 30% acetonitrile (MeCN) vs. 0.1% HCl in H₂O. For acetate exchange, peptides can be loaded from the same diluted TFA solution, washed with 3-4 column volumes of 1% acetic acid (AcOH) in H₂O, followed by 2 column volumes of 0.1 M NH₄OAc in H₂O, pH 4.4. In some embodiments, the column is washed again with 3-4 column volumes of 1% AcOH in H₂O.

Peptides can be eluted from the columns using a step gradient of 30% MeCN vs. 1% AcOH in H₂O. In some embodiments, the elution of peptides can be enhanced by acetate exchange. Exemplary buffers for acetate exchange include 0.1 M NH₄OAc in H₂O, pH 4.4.

Analytical HPLC can be carried out to assess the purity and homogeneity of peptides. An exemplary HPLC column for use in analytical HPLC is a PHENOMENEX® JUPITER® column. In some embodiments, analytical HPLC is carried out using a column and buffer that are heated to a temperature greater than 25° C., for example 25-75° C. In a particular embodiment, analytical HPLC is carried out at temperatures of about 65° C. A step gradient can be used to separate the peptide composition. In some embodiments, the gradient is from 1%-40% MeCN vs 0.05% TFA in H₂O. The change in gradient can be achieved over 20 minutes using a flow rate of 1 ml/min. Peptides can be detected using UV detection at 215 nm.

Where the peptides or compositions thereof are required to be sterilized or otherwise processed for the removal of undesirable contaminants and/or micro-organisms, filtration can be used. Filtration can be achieved using any system or procedures known in the art. In some embodiments, filtration removes contaminants or prevents the growth or presence of microorganisms. Exemplary microorganisms and contaminants that can be removed include bacteria, cells, protozoa, viruses, fungi, and combinations thereof. In some embodiments, the step of filtration is carried out to remove aggregated or oligomerized peptides. For example, solutions of the peptides can be filtered to remove assembled peptide structures or oligomers on the basis of size.

The present invention will be further understood by reference to the following non-limiting examples.

### EXAMPLES

### Example 1: A Decoy Peptide Attenuates eNOS Mediated Mitochondrial Dysfunction and Symptoms of Ventilator-Induced Lung Injury

Materials and Methods

Cell Culture

Primary cultures of ovine pulmonary arterial endothelial cells (PAEC) were isolated as described previously (L. K. Kelly, Am J Physiol Lung Cell Mol Physiol, 286(5), L984-91 (2004)). Briefly, cells were maintained in Dulbecco's modified Eagle medium (DMEM) supplemented with 10% fetal calf serum (Hyclone, Logan, UT), antibiotics/antimycotic (500 IU Penicillin, 500 μg/ml Streptomycin, 1.25 μg/ml Amphotericin B; MediaTech, Herndon, VA) at 37° C. in a humidified atmosphere with 5% CO₂ and 95% air. Cells were used for experiments between passages 9-14, seeded at ˜50% confluence, and utilized when fully confluent.

Mouse Model of VILI

Male C57BL/6 mice aged between 6 and 8 weeks were purchased from Jackson Laboratories (ME, USA). Mice were maintained at a room temperature of 22±1° C. in air with 40-70% humidity at least one week before experiments. Animals were randomly distributed into 4 groups (n=5 in each group): non-ventilated control mice with normal saline; non-ventilated control mice treated with the eNOS decoy peptide (d-peptide); high tidal volume with normal saline; high tidal volume with eNOS decoy peptide. Three hours before ventilation, saline or the eNOS decoy peptide (10 mg/kg body weight) were injected intraperitoneally. Before mechanical ventilation was initiated, mice were anesthetized using an intraperitoneal injection with a cocktail containing ketamine (100 mg/kg) and xylazine (5 mg/kg). The mice were then placed in a supine position on a heating pad to maintain body temperature. For the ventilation procedure, mice were orotracheally intubated with a 20 g intravenous indwelling catheter and attached to a small animal ventilator (SAR-1000, CWE Inc., USA). The ventilation parameters were set as follows: inspiration/expiration ratio, 33%; respiratory rate, 75 breaths/min; and tidal volume, 35 mL/kg (high tidal volume group). During mechanical ventilation, mice were maintained in deep anesthesia by injecting with ketamine (100 mg/kg) every 45 minutes for the duration of the 4 h study. Mice in the non-ventilated control group were allowed to spontaneously breathe. At the end of the study period, 1 ml of pre-chilled PBS was used to flush the lungs through the tracheal cannula and the resulting bronchial alveolar lavage fluid (BALF) was collected and centrifuged at 500× g for 10 min at 4° C. The pellets were then resuspended in 500 μl of PBS and the cell numbers present were determined using an automated cell counter. The BAL fluid was centrifuged again at 15,000× g at 4° C. for 15 min and the supernatant collected and stored at −80° C. until the protein concentration was measured. After BALF collection, the mice were sacrificed immediately, and lungs were collected and frozen in liquid nitrogen for Western blot analysis. All animal procedures were approved by the Animal Care and Use Committee of the University of Arizona.

Antibodies and Chemicals

Mouse eNOS antibody, BD Transduction laboratories (San Jose, CA), Cat #610296. Mouse eNOS (pT495) antibodies, BD Transduction laboratories (San Jose, CA), Cat #612706. Mouse β-actin antibody, Sigma (St. Louis, MO), Cat #A1978-200 UL. Rabbit PKCα antibody, rabbit Phospho-(Ser) PKC antibody, Cell Signaling (Danvers, MA), Cat #2056S. Mouse eNOS polyclonal antibody, ThermoFisher (Waltham, MA), Cat #PA3-031A. MitoTracker, Invitrogen (Carlsbad, CA), Cat #7512. MitoSOX Red, Molecular Probes (Eugene, OR). TMRM (tetramethylrhodamine methyl ester perchlorate), Molecular Probes (Eugene, OR), Cat #134361. Dihydrorhodamine 123, EMD Millipore (Billerica, MA), Cat #D1054. Goat Anti-Mouse/Rabbit Cy2 antibody and Goat Anti-Mouse/Rabbit Cy3 antibody, Jackson ImmunoResearch (West Grove, PA). 4αPDD (4α-Phorbol-12,13 didecanoate), Millipore (Billerica MA), Cat #524394-1 MG. PMA (Phorbol 12-myristate 13-acetate), Sigma-Aldrich (St. Louis, MO), Cat #P1585.

Measurement of Cytosolic Ca²⁺ Concentration ([Ca²⁺]Cyt)

PAEC were grown at 50-60% confluence on 25-mm-diameter circular glass coverslips. Cells were first incubated with 4 μM fura-2 acetoxymethyl ester (fura-2/AM; Invitrogen/Molecular Probes, Eugene, OR) in HEPES-buffered solution for 60 mM at room temperature (22-24° C.) and then superfused with the HEPES-buffered solution for 30 mM to washout residual extracellular fura-2/AM and allow sufficient time for intracellular esterase to cleave AM from fura-2/AM. Cells loaded with fura-2 were alternatively illuminated at 340 and 380 nm wavelengths by a xenon lamp (Hamamatsu Photonics, Hamamatsu, Japan) connected to an inverted fluorescent microscope (Eclipse Ti-E; Nikon, Tokyo, Japan). The fluorescence emission (at 520 nm) was captured with an EM-CC camera (Evolve; Photometric, Tucson, AZ) and analyzed using NIS Elements 3.2 software (Nikon). [Ca²⁺]cyt is expressed as 340/380 fluorescence ratio within an area of interest in the peripheral area of a cell recorded every 2 s. The 340/380 ratio was used to calculate the [Ca²⁺]cyt in nanomolar concentration. [Ca²⁺]cyt was calculated using the following equation: [Ca²⁺]cyt=Kd×(Sf2/Sf1)×(R−Rmin)/(Rmax−R). Kd (225 nM) is the dissociation constant of the Ca2+-fura-2 complex; and Sf2 and Sf1 and Rmin and Rmax were calculated using a standard protocol (G. Grynkiewicz, J Biol Chem, 260(6), 3440-50 (1985)). The HEPES-buffered solution contained (in mM) 137 NaCl, 5.9 KCl, 1.8 CaCl2, 1.2 MgCl2, 14 glucose, and 10 HEPES (pH was adjusted to 7.4 with 10 N NaOH). The Ca²⁺-free solution was prepared by replacing 1.8 mM CaCl₂ with equimolar MgCl₂ and adding 0.1 mM EGTA to chelate residual Ca²⁺. All experiments for measurement of [Ca²⁺]cyt were carried out at room temperature (22-24° C.).

Measurement of eNOS Derived Superoxide

Superoxide levels were estimated by electron paramagnetic resonance (EPR) assay using the spin-trap compound 1-hydroxy-3-methoxycarbonyl-2,2,5,5-tetramethylpyrrolidine HCl (CMH, Axxora) as described previously[18, 19]. Superoxide in PAEC was trapped by incubating PAEC with 20 μl of CMH stock solution (20 mg/ml) for 1 h, followed by trypsinization and centrifugation at 500 g. The cell pellet was suspended in 35 μl DPBS and loaded into a capillary tube which was then analyzed with a MiniScope MS200 EPR machine (Magnettech, Berlin, Germany). Pre-incubating cells or tissue with 100 μM ethylisothiourea (ETU, Sigma-Aldrich) for 30 min followed by incubation with CMH measured NOS-derived superoxide. EPR spectra were analyzed using ANALYSIS v.2.02 software (Magnettech). Differences between levels of samples incubated in the presence and absence of ETU were used to determine NOS-dependent superoxide generation.

Determination of Mitochondrial Reactive Oxygen Species (ROS) Levels

MitoSOX™ Red (Molecular Probes), a fluorogenic dye for selective detection of ROS levels in the mitochondria of live cells was used. Briefly, cells were washed with fresh media, and then incubated in media containing MitoSOX Red (2 μM), for 30 min at 37° C. in dark conditions then subjected to fluorescence microscopy at an excitation of 510 nm and an emission at 580 nm. An Olympus IX51 microscope equipped with a CCD camera (Hamamatsu Photonics) was used for acquisition of fluorescent images. The average fluorescent intensities (to correct for differences in cell number) were quantified using ImagePro Plus version 5.0 imaging software (Media Cybernetics, Rockville, MD).

Measurement of Peroxynitrite Levels

The level of cell peroxynitrite was determined by the oxidation of dihydrorhodamine (DHR) 123 (EMD Millipore, Billerica, MA) to rhodamine 123, as previously described (S. Aggarwal, J Cell Physiol, 226(12), 3104-13 (2011)). Briefly, cultured PAEC were treated with or without TGF-β1 (5 ng/ml, 8 h) or GW9662 (5 μM, 24 h). The cells were collected and the cell pellet was then treated with PEG-Catalase (100 U, 30 min) to reduce H₂O₂ dependent DHR 123 oxidation. DHR 123 (5 μM, 30 min) was added to the cell pellet in phenol red-free media and the fluorescence of rhodamine 123 measured using a Fluoroskan Ascent Microplate Fluorometer with excitation at 485 nm and emission at 545 nm. Fluorescent values were normalized to the protein levels in each sample.

Analysis of Mitochondrial Membrane Potential

Mitochondrial membrane potential was determined using TMRM (tetramethylrhodamine methyl ester perchlorate, Molecular Probes, Eugene, OR). Briefly, after each experiment, cells were washed with fresh media, incubated in media containing TMRM (50 nM), for 30 min at 37° C. in dark conditions, then subjected to fluorescence microscopy using an excitation of 548 nm and an emission at 575 nm. An Olympus IX51 microscope equipped with a CCD camera (Hamamatsu Photonics) was used for acquisition of fluorescent images and the average fluorescent intensities were quantified using ImagePro Plus version 5.0 imaging software (Media Cybernetics).

Analysis of Mitochondrial Bioenergetics

The XF24 Analyzer (Seahorse Biosciences) and XF CELL MITO STRESS™ Test Kit (#101706-100; Seahorse Biosciences) were used for the mitochondrial bioenergetic analyses. The optimum number of cells/well was determined to be 75,000/0.32 cm². At the end of each study, the XF24 culture microplates were incubated in a CO₂-free XF prep station at 37° C. for 45 min to allow temperature and pH calibration. Subsequently, each well was sequentially injected Oligomycin (1 μM final concentration), carbonyl cyanide 4-(trifluoromethoxy) phenylhydrazone (FCCP, 1 μM final concentration), and Rotenone+antimycin A (1 μM final concentration of each) and measured the oxygen consumption rate (OCR). These agents were used to determine basal mitochondrial respiration, reserve respiratory capacity and maximal respiratory capacity measurements in pmols/min of oxygen consumed.

Western Blot Analysis

Protein extracts were prepared using lysis buffer (50 mM Tris-HCl, pH 7.6, 0.5% Triton X-100, 20% glycerol) containing HALT™ protease inhibitor cocktail (Pierce Laboratories, Rockford, IL). The extracts were then subjected to centrifugation (15,000 g for 15 min at 4° C.). Supernatant fractions were assayed for protein concentration using the Bradford reagent (Bio-Rad, Richmond, CA) then used for Western blot analyses. Protein extracts (25-50 μg) were separated on Long-Life 4-20% Tris-SDS-Hepes gels and electrophoretically transferred to IMMUNO-BLOT™ PVDF membrane (Bio-Rad Laboratories, Hercules, CA) Immunoblotting was then carried out using the appropriate antibodies in Tris-base buffered saline with 0.1% Tween 20 and 5% nonfat milk. After washing, the membranes were probed with horseradish peroxidase-conjugated goat antiserum to rabbit or mouse. Reactive bands were visualized using chemiluminescence (Super Signal West Femto; Pierce, Rockford, IL) on a LI-COR Odyssey image station (Lincoln, NE). Bands were quantified using LI-COR Image Station software. Loading was normalized by reprobing the membranes with an antibody specific to β-actin.

Immunofluorescent Microscopy

PAEC were grown on cover glass for three days after reaching 100% confluence, and fixed with 4% paraformaldehyde (Thermo Fisher Scientific) for 30 min, permeabilized with 100% cold methanol at −20° C. for 5 min Cell then blocked with 1% BSA for 1 hour, and later incubated with first antibody overnight at 4° C. then secondary antibody at room temperature for 1 hour. Finally, cells were mounted on microscope slides using ProLong Glass Antifade Mountant, (Invitrogen, Carlsbad, CA, Cat #P36980). Immunofluorescent images were observed with a Nikon Eclipse TE2000-U microscope, with Hamamatsu digital camera C11440, and Olympus IX51 microscope with Hamamatsu digital camera C4742-95. The images were analyzed with ImagePro Plus 7.0 (W. Casavan, Microscopy Today, 11(6), 48-50 (2003)) or ImageJ software to evaluate the colocalization of fluorescent.

Measurement of PKC Activity

Activation of PKC in control and 4αPDD-treated EC was evaluated using dot-blot method and antibodies specific to PKC-phosphorylated proteins. The cell lysates were transferred on nitrocellulose membrane using Bio-Dot Microfiltration apparatus (Bio-Rad) according to Instruction Manual. To detect and evaluate levels of PKC-phosphorylated proteins, Phospho-(Ser) PKC Substrate rabbit polyclonal antibody (Cell Signaling) was used. After visualization of PKC-phosphorylated proteins, the membrane was stripped and re-probed with β-actin antibody for normalization.

Transient Transfections

The constitutively active PKCα mutant, myr-PKCα was purchased from Origene (Rockville, MD) and purified using an endotoxin free kit (Qiagen, USA). PAEC cultured to 80% confluence were then transiently transfected using the Effectene Transfection Reagent (Qiagen, USA) according the manufacturers protocol.

In Vitro Peptide Binding Assay

Biotinylation of the decoy peptide (d-peptide) was performed using the Thermo Scientific EZ-Link Sulfo-NHS-LC-Biotinylation Kit. Briefly, Sulfo-NHS-LC-Biotin was mixed with d-peptide and the reaction mix was incubated at room temperature for 30 minutes. Different concentrations of the biotinylated d-peptide (0 to 5 μg) mixed with either purified eNOS protein or PKCα protein in a reaction mix containing PKC lipid activator were incubated at room temperature for 1 hour. Protein bound to the biotinylated d-peptide was captured using Thermo Scientific streptavidin agarose column and run on a 10% SDS-PAGE gel under reducing condition. The resulting blots were probed with antibodies to eNOS and PKCα protein respectively. Reactive bands were visualized using chemiluminescence on the LI-COR Odyssey image station.

Measurement of Trans Endothelial Resistance (TER)

Transendothelial Electrical Resistance (TER) was determined to characterize the integrity of PAEC monolayers using an electrical cell-substrate impedance sensing (ECIS) instrument ECIS Z-Theta (Applied BioPhysics, Troy, NY) as previously described (J. N. Gonzales, Vascul Pharmacol, 62(2), 63-71 (2014); S. Aggarwal, American Journal of Respiratory Cell and Molecular Biology, 50(3), 614-25 (2014)). The cells were plated in 8-well ECIS arrays (Applied BioPhysics) in complete cell culture medium (DMEM supplemented with 10% FBS) and grown to 100% confluency for 2 days. Then, cell culture medium was changed for fresh one, and the EC were used in TER assay. Initial resistance at the onset of our experiments was 900 to 1000 in array wells, and then all wells were normalized to 1. 4000-Hz AC signal with 1-V amplitude was applied to the EC monolayers through a 1-M-Ω resistor, creating an approximate constant-current source (1 μA). After a baseline measurement, the EC were treated with 4αPDD, PMA, the eNOS pT495 decoy peptide, or vehicle at the indicated concentrations, and changes in TER were recorded in real time.

Statistical Analysis

Statistical calculations were performed using the GrapPad Prism software. The mean±SEM was calculated for all samples. Statistical significance was determined either by the unpaired t-test (for 2 groups) or ANOVA (for ≥3 groups) with Newman-Keuls post-hoc testing. A value of P<0.05 was considered significant.

Results

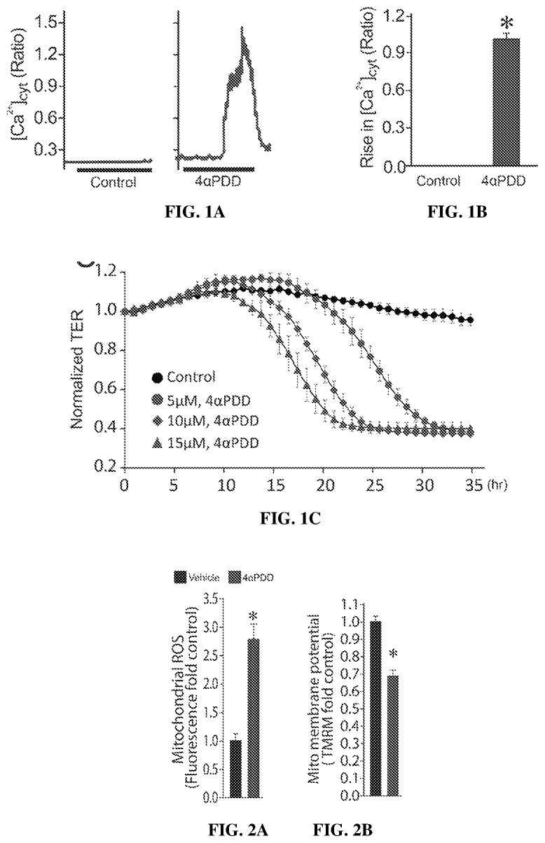

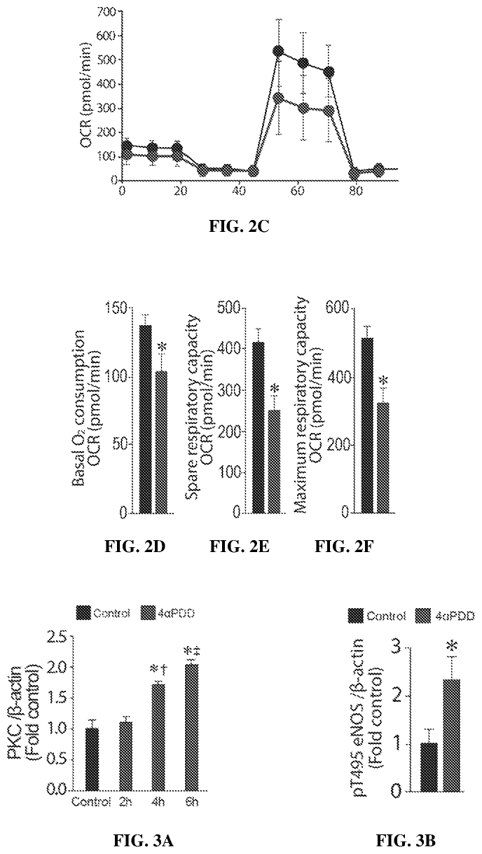

Increase in Permeability Induced by 4αPDD is Associated with Disruption of Mitochondrial Function in PAECs

[Ca²⁺]cyt measurements performed with fura-2/AM loaded PAEC demonstrated that 4αPDD exposure induced a transient increase of [Ca²⁺]cyt via Ca²⁺ influx (FIG. 1A-1B). The increase in [Ca²⁺]cyt correlated with a dose-dependent decrease in TER, indicating a disruption of barrier integrity (FIG. 1C). The decrease in TER induced by TRPV4 activation correlated with the disruption of mitochondrial function as determined by increases in mitochondrial ROS levels, estimated by increases in MitoSOX red fluorescence (FIG. 2A, 2B) and a decrease in the mitochondrial membrane potential, evaluated using the probe tetramethyl rhodamine methyl ester (TMRM, FIG. 2A, 2B). Effects on mitochondrial bioenergetics were also measured (FIG. 2C). The data indicate that 4αPDD disrupts bioenergetics as determined by reductions in mitochondrial basal O₂ consumption, spare respiratory capacity and maximum respiratory capacity (FIG. 2D-2F).

4αPDD-Mediated Disruption of Mitochondrial Bioenergetics is Associated with Mitochondrial Redistribution of Uncoupled eNOS

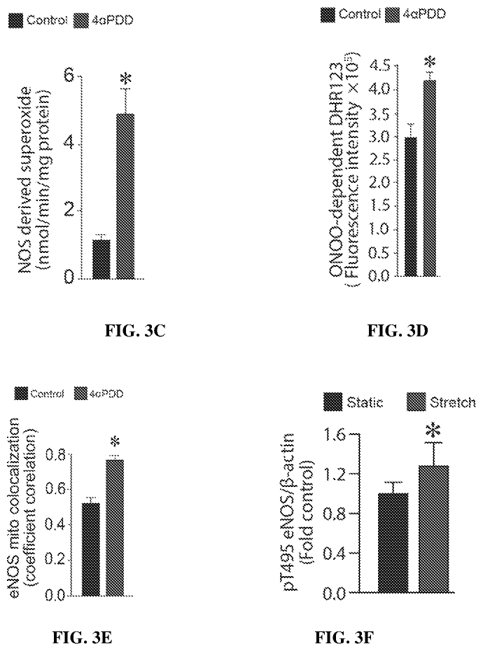

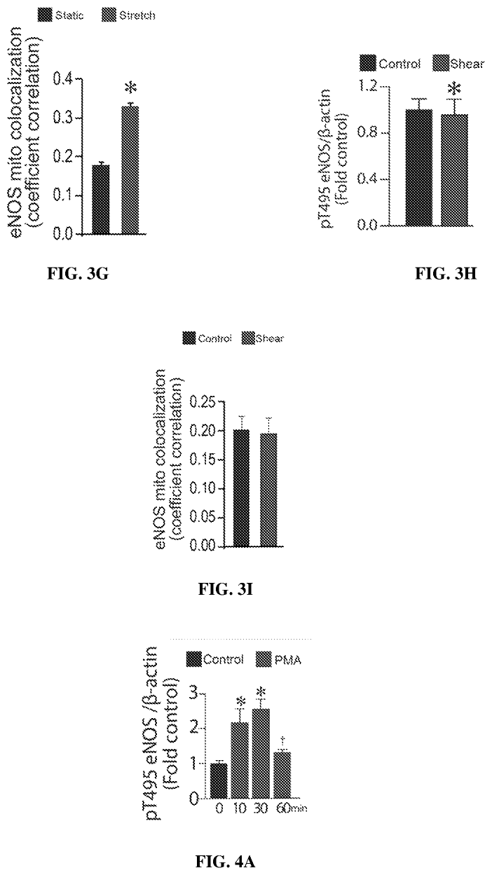

Phosphorylation of eNOS at T495 by PKC results in its uncoupling and mitochondrial redistribution (X. Sun, American Journal of Respiratory Cell and Molecular Biology, 50(6), 1084-95 (2014)). Thus, it was investigated whether this was the mechanism by which TRPV4 activation disrupts mitochondrial function. The data indicate that the increase in intracellular [Ca²⁺] associated with 4αPDD exposure (FIG. 1A) increases PKC activity in PAEC (FIG. 3A). This results in an increase in eNOS phosphorylation at T495 (FIG. 3B) and eNOS uncoupling as determined by increases in NOS derived superoxide generation (FIG. 3C) and cellular peroxynitrite levels (FIG. 3D) Immunofluorescence microscopy confirmed that 4αPDD exposure induces the mitochondrial redistribution of eNOS (FIG. 3E). Further, it was observed that cyclic stretch mimicked the effect of 4αPDD in PAEC, increasing both pT495-eNOS levels (FIG. 3F) and the mitochondrial redistribution of eNOS (FIG. 3G). As laminar shear stress did not increase either pT495-eNOS levels (FIG. 3H) or the mitochondrial redistribution of eNOS (FIG. 3I), these data demonstrate differential effects of mechanical forces on eNOS phosphorylation and sub-cellular redistribution.

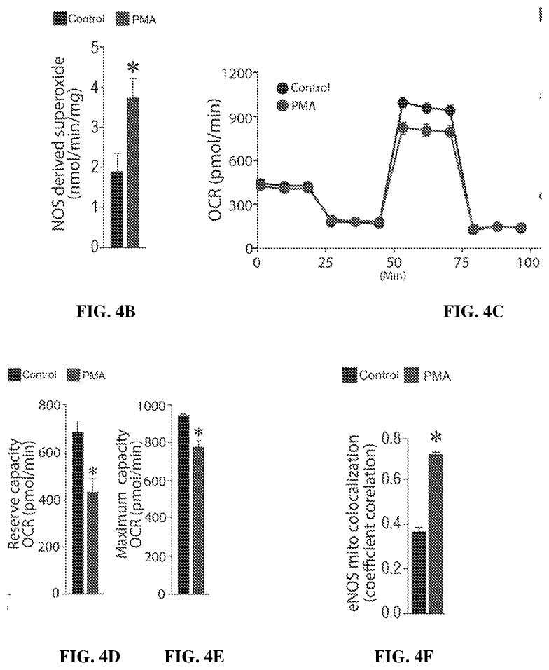

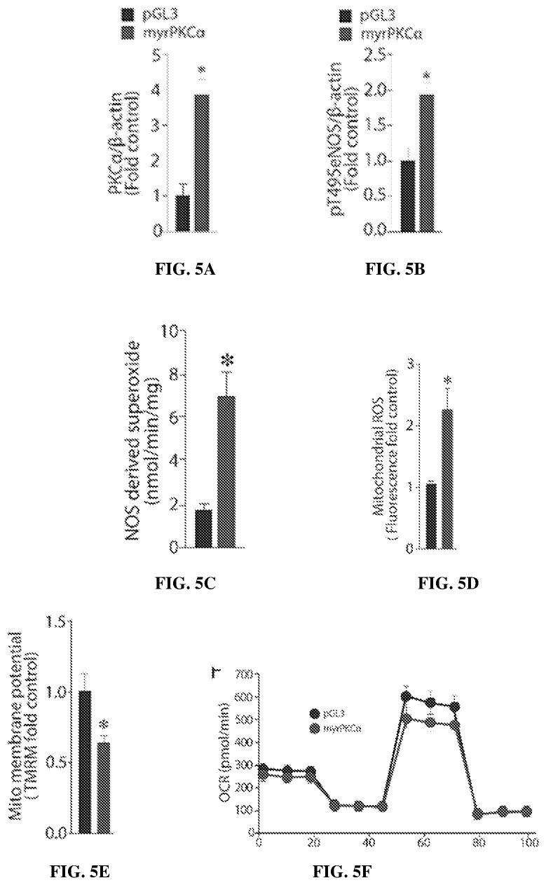

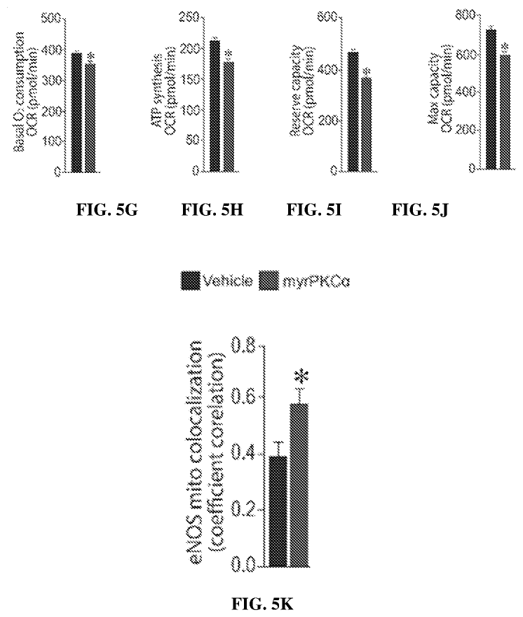

To confirm the role of PKC in the phosphorylation of eNOS at T495 and the disruption of mitochondrial function, PAECs were exposed to the PKC activator, phorbol myristate acetate (PMA). PMA mimicked the effects of 4αPDD, resulting in increases in pT95-eNOS levels (FIG. 4A), eNOS uncoupling (FIG. 4B), and the disruption of mitochondrial bioenergetics (FIG. 4C-D). PMA exposure also induced the mitochondrial redistribution of eNOS (FIG. 4E). The over-expression of a constitutively active mutant of PKCα alone (FIG. 5A) was able to recapitulate the action of 4αPDD stimulating eNOS phosphorylation at T495 (FIG. 5B), eNOS uncoupling (FIG. 5C) while increasing mitochondrial ROS levels (FIG. 5D) and decreasing the mitochondrial membrane potential (FIG. 5E). Mitochondrial bioenergetics were also disrupted (FIGS. 5F-G) and the mitochondrial redistribution of eNOS was increased (FIG. 5H-K).

Blocking eNOS Phosphorylation at T495 Attenuates Injury Associated with Mechanical Ventilation of the Mouse Lung

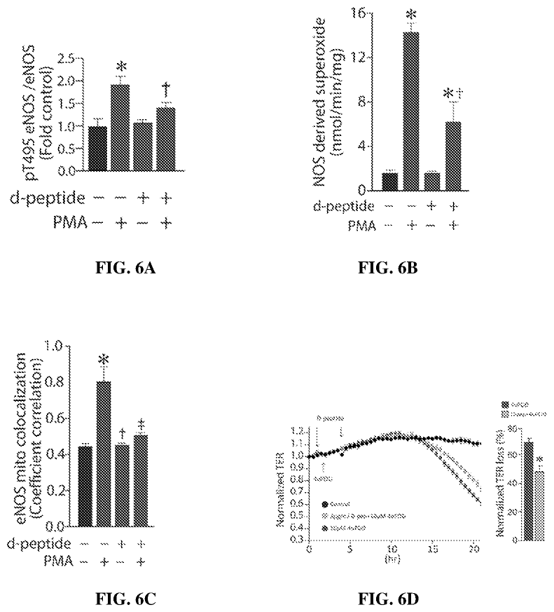

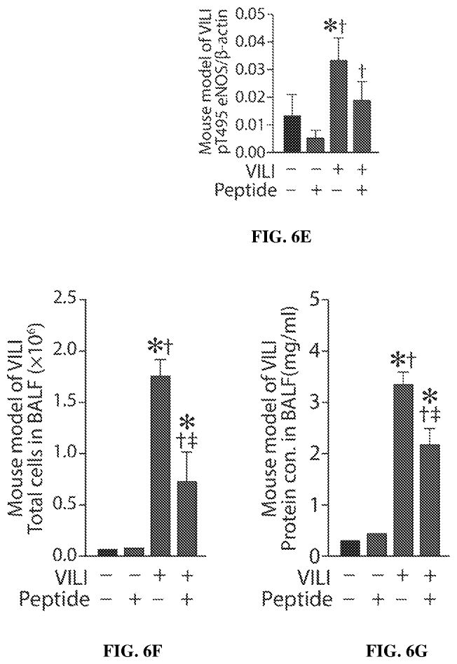

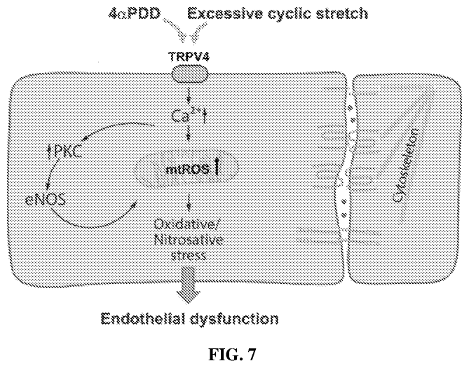

To further investigate the role of eNOS phosphorylation at T495 in the increase in permeability induced by TRPV4 activation, a decoy peptide (d-peptide) was developed to prevent eNOS T495 phosphorylation. The peptide, sequence HRKKRRQRRITRKKTFKEVA (SEQ ID NO:1), was first tested for specificity using an in vitro binding assay. It was observed that the d-peptide binds efficiently to purified PKCα but not to eNOS), indicating it acts as a decoy of eNOS for PKC. When introduced into PAEC, the d-peptide attenuated the PMA-mediated increase in pT495-eNOS (FIG. 6A) and reduced eNOS uncoupling (FIG. 6B). The PMA-induced mitochondrial redistribution of eNOS was attenuated (FIG. 6C) and the barrier disruption induced by 4αPDD was reduced (FIG. 6D). Finally, the d-peptide attenuated the increase in pT495-eNOS induced by mechanical ventilation of the mouse lung (FIG. 6E) and this correlated with a reduction in VILI as demonstrated by decreases in the cell number (FIG. 6F) and protein levels (FIG. 6G) in the BALF indicative of decreased pulmonary capillary permeability.

Ventilator-induced lung injury (VILI) is the consequence of acute lung injury (ALI) that occurs with the use of mechanical ventilation (“International consensus conferences in intensive care medicine: Ventilator-associated Lung Injury in ARDS,” Am. J. Respir. Crit. Care. Med., 160(6):2118-24 (1999)). VILI is indistinguishable morphologically, physiologically, and radiologically from the diffuse alveolar damage seen in ALI (Am. J. Respir. Crit. Care. Med., 160(6):2118-24 (1999). VILI is a significant problem with the use of mechanical ventilation to treat ARDS. Mechanical ventilation itself can also injure the lungs even when ALI or ARDS is not initially present (O. Gajic, Intensive Care Med, 31(7), 922-6 (2005); O. Gajic, Crit Care Med, 32(9), 1817-24 (2004); R. M. Determann, Crit Care, 14(1), R1 (2010)).

The current standard of care for ALI/ARDS uses protective lung ventilation strategies (D. Dreyfuss, The American Review of Respiratory Disease, 137(5), 1159-64 (1988); H. H. Webb, The American Review of Respiratory Disease, 110(5), 556-65 (1974); A. S. Slutsky, American Journal of Respiratory and Critical Care Medicine, 163 (3 Pt. 1) 599-600 (2001)). These ventilator strategies are based on the ARDS network trial (Acute Respiratory Distress Syndrome Network, et al., N. Engl. J. Med., 342(18):1301-8 (2000)). However, these protective ventilation strategies are supportive and not therapeutic. Thus, there is intense interest in understanding the molecular mechanisms by which VILI leads to the development of ARDS. One of the major areas of investigation in VILI is the mechanical force dependent activation of transient receptor potential (TRP) channels which are permeable to Ca²⁺ since aberrant Ca²⁺ entry is one of the most widely acknowledged mechanisms that induce endothelial permeability (F. E. Curry, FASEB Journal, 6(7), 2456-66 (1992); C. Tiruppathi, Vascul Pharmacol, 39(4-5), 173-85 (2002)). In mammals, 28 TRP channel isoforms have been identified which are divided into six subfamilies, TRPA, TRPC, TRPM, TRPML, TRPP and TRPV. They present a common feature of a tetrameric structure, four subunits forming the pore of the channel and a ring of four negative charged residues at the external end of the pore composing the selectivity filter (M. G. Madej, Pflugers Arch, 470(2), 213-225 (2018); T. Hof, Cardiology, 16(6), 344-360 (2019)). TRPV4 has been identified as a key Ca²⁺ channel (J. P. White, Physiological Reviews, 96(3), 911-73 (2016)) and is activated by physical stimuli such as mild heat, hypoosmotic conditions, and membrane deformation (K. Venkatachalam, Annu Rev Biochem, 76, 387-417 (2007)). TRPV4 activation and Ca²⁺ entry can also occur by mechanical stimulation, and the data show that the exposure of PAEC to cyclic stretch induces eNOS phosphorylation and eNOS mitochondrial redistribution in a similar manner to the direct activation of TRPV4 using 4α-phorbol didecanoate (4αPDD). The exposure of PAEC to laminar shear stress for the same duration did not induce eNOS phosphorylation or eNOS mitochondrial redistribution. This supports reports that the endothelium responds differently depending on the mechanical force to which it is exposed. However, the literature is unclear. Thus, mechanical stress has been shown to both uncouple eNOS (K. Vaporidi, Am J Physiol Lung Cell Mol Physiol, 299(2), L150-9 (2010)) and stimulate NO generation (Z. Hu, PLoS One, 8(8), e71359 (2013)) from eNOS. This is likely due to differential effects on ECs from different ages, vascular beds, and potentially, to both the duration and level of the mechanical force utilized (K. Vaporidi, Am J Physiol Lung Cell Mol Physiol, 299(2), L150-9 (2010); S. Wedgwood, Am J Physiol Lung Cell Mol Physiol, 284(4), L650-62 (2003); S. M. Black, Am J Physiol, 275(5), H1643-51 (1998); S. M. Black, J Clin Invest, 100(6), 1448-58 (1997)). In addition, although laminar shear stress predominantly stimulates eNOS activity and NO release (S. A. Kim, Biochem Biophys Res Commun, 490(4), 1369-1374 (2017); K. Binti Md Isa, Biochem Biophys Res Commun, 412(2), 318-22 (2011); J. Tian, Free Radic Biol Med, 49(2), 159-70 (2010); G. K. Kolluru, Nitric Oxide, 22(4), 304-15 (2010); S. Kumar, Am J Physiol Lung Cell Mol Physiol, 298(1), L105-16 (2010)), oscillatory flow uncouples eNOS (K. L. Siu, J. Biol Chem, 291(16), 8653-62 (2016)). Thus, different types of mechanical forces can act differently on regions of the vascular wall to affect NO bioavailability and potentially contribute to disease pathogenesis. At least acutely, could be dependent on which phosphorylation site on eNOS is regulated such that increasing pS1177-eNOS levels will be associated with eNOS activation and NO generation (R. Sathanoori, Cell Mol Life Sci, 74(4), 731-746 (2017)) while increases in pT495-eNOS will be associated with eNOS inactivation and uncoupling (F. Chen, PLoS One, 9(7), e99823 (2014); X. Sun, American Journal of Respiratory Cell and Molecular Biology, 50(6), 1084-95 (2014); S. Ghosh, Am J Physiol Lung Cell Mol Physiol, 310(11), L1199-205 (2016)).

High vascular pressure and ventilator-induced lung injury have both been reported to increase lung endothelial permeability by promoting Ca²⁺ entry via TRPV4 (M. Y. Jian, American Journal of Respiratory Cell and Molecular Biology, 38(4), 386-92 (2008); K. Hamanaka, Am J Physiol Lung Cell Mole Physio, 293(4), L923-32 (2007)) and 4αPDD exposure also leads to Ca²⁺ entry-dependent acute lung injury, disruption of the lung barrier, and alveolar flooding (D. F. Alvarez, Circ Res, 99(9), 988-95 (2006)). Conversely, 4αPDD does not increase lung permeability in TRPV4-knockout mice (D. F. Alvarez, Circ Res, 99(9), 988-95 (2006)). However, beyond Ca²⁺-mediated cytoskeleton rearrangements (C. Tiruppathi, Circulation Research, 91(1), 70-6 (2002)) the mechanisms by which TRPV4 activation induces EC permeability are unresolved. Thus, the results add significantly to knowledge regarding TRPV4 mediated EC permeability by demonstrating an important role for PKC-mediated phosphorylation and uncoupling of eNOS in the development of VILI. Recent studies have demonstrated that TRPV4^{-/-} mice or mice treated with the TRPV4 antagonist, GSK2193874, are protected against acid-induced ALI (J. Yin, Am J Respir Cell Mol Bio, 54(3), 370-83 (2016)). TRPV4 inhibition was only protective if given in a preventative manner (J. Yin, Am J Respir Cell Mol Bio, 54(3), 370-83 (2016)). This lack of a therapeutic window indicates that the downstream targets of TRPV4 may be more viable targets for therapy. Indeed, the data demonstrating that targeting eNOS phosphorylation at T495 using a decoy peptide attenuates VILI in a mouse model of mechanical stretch, validates this approach and opens up a new avenue for treating/preventing VILI in humans. It is contemplated that the d-peptide can be modified to increase its stability and/or be linked with a delivery system that will specifically target the damaged endothelium. ARDS cases stratified according to disease severity have been shown to be associated with VILI in 48.8% of the entire patient population, 87% in late ARDS, 46% in intermediate ARDS, and 30% in early ARDS (L. Gattinoni, JAMA, 271(22), 1772-9 (1994)).

The identification of a role for eNOS uncoupling in TRPV4 mediated EC barrier disruption is in agreement with work showing that eNOS is an important source of ROS in VILI (K. Vaporidi, Am J Physiol Lung Cell Mol Physiol, 299(2), L150-9 (2010)). As eNOS uncoupling is associated with ALI in gram positive (F. Chen, PLoS One, 9(7), e99823 (2014)) and gram negative bacteria (C. M. Gross, PloS One, 10(3), e0119918 (2015)) exposure models as well a smoke inhalation and burn injury models (K. Murakami, Shock, 28(4), 477-83 (2007)) it is likely a common mechanism for the development of ALI induced by multiple stimuli. However, the mechanism by which eNOS becomes uncoupled can be different. The data implicate T495 phosphorylation in eNOS uncoupling in VILI and gram positive sepsis (F. Chen, PLoS One, 9(7), e99823 (2014)) while in gram negative sepsis-eNOS uncoupling involves increases in the levels of the endogenous NOS uncoupler, asymmetric dimethlyarginine (ADMA) (S. Aggarwal, American Journal of Respiratory Cell and Molecular Biology, 50(3), 614-25 (2014); S. Sharma, Vascul Pharmacol, 52(5-6), 182-90 (2010)). Mechanical ventilation is associated with the oxidation of tetrahydrobiopterin (BH4) to BH2 (K. Vaporidi, Am J Physiol Lung Cell Mol Physiol, 299(2), L150-9 (2010)) and important NOS co-factor that is required for efficient enzymatic coupling (U. Forstermann, Eur Heart J, 33(7), 829-37, 837a-837d (2012); R. Rafikov, J Endocrinol, 210(3), 271-84 (2011); M. J. Crabtree, Nitric Oxide 25(2), 81-8 (2011)). As BH2 itself can increase eNOS uncoupling (A. C. Grobe, Lung Cellular and Molecular Physiology, 290(6), L1069-77 (2006)), it is possible that increases in BH2 could synergize with T495 phosphorylation to further increase eNOS uncoupling.

In pulmonary hypertension (PH), an endothelin-1 (ET-1) mediated increase in PKCδ activity induces the mitochondrial redistribution of eNOS through increased phosphorylation of eNOS at T495 (X. Sun, American Journal of Respiratory Cell and Molecular Biology, 50(6), 1084-95 (2014)) and that increased peroxynitrite generation is a prerequisite for the mitochondrial redistribution of uncoupled eNOS (R. Rafikov, J. Biol Chem, 288(9) 6212-26 (2013)). As the data demonstrates that T495 phosphorylation induces eNOS uncoupling and peroxynitrite generation, it is possible that the phosphorylation of T495 is a common mechanism by which kinases can stimulate the mitochondrial redistribution of eNOS. Since Rho-kinase (ROCK) phosphorylates eNOS at T495 (J. Seo, Free Radic Biol Med, 90, 133-44 (2016)) and is also intimately involved in the development of ALI (F. Abedi, Pharmacol Res, 155, 104736 (2020)), ROCK signaling may also induce EC barrier disruption through increases in T495 phosphorylation. However, it should also be noted that the mitochondrial redistribution of eNOS can also be induced by its phosphorylation at 5635 by Akt1 (R. Rafikov, J Biol Chem, 288(9), 6212-26 (2013); X. Sun, Am J Respir Cell Mol Biol, 55(2), 275-87 (2016)). However, in this case, eNOS appears to enhance mitochondrial function as a S635D-eNOS mutant reduces the mitochondrial OCR and reduces mitochondrial ROS levels (R. Rafikov, J Biol Chem, 288(9), 6212-26 (2013)). As it is becoming more accepted that Akt1 is involved in the resolution phase of ALI (T. Wang, Am J Physiol Lung Cell Mol Physiol, 312(4), L452-L476 (2017)), it is contemplated that mitochondrial redistributed eNOS due to phosphorylation at 5635 could reduce mitochondrial ROS and perhaps mitochondrial function. This could be important due to the key role played by mitochondrial ROS in the inflammatory response via the activation of the inflammasome. Although important for the clearance of pathogens during bacterial infection, sustained or excessive inflammasome activation may exacerbate pathological inflammation (B. K. Davis, Annu Rev Immunol, 29, 707-35 (2011)). Inflammasomes are a group of cytosolic protein complexes that regulate the activation of caspase-1, and the processing of pro-interleukin (IL)-113 and pro-IL-18 to their mature active forms (F. Martinon, Mol Cell, 10(2), 417-26 (2002)). The activation of the NLRP3 inflammasome is a two-step process: the expression of NLRP3 and pro-IL-1β is induced by transcriptional up-regulation via NF-κB signaling (F. G. Bauernfeind, J Immunol, 183(2), 787-91 (2009)) followed by the assembly of NLRP3 inflammasome protein components in order to form a platform to activate caspase-1. Caspase 1 is then able to cleave pro-IL-1β and pro-IL-18 allowing them to be secreted from cells (F. Martinon, Mol Cell, 10(2), 417-26 (2002)). As one of the mechanisms identified for NLRP3 inflammasome assembly is the generation of mitochondrial ROS (A. Abderrazak, Redox Biol, 4, 296-307 (2015)), it is likely that the mitochondrial redistribution of pT495-eNOS is involved in the activation of the inflammasome while the mitochondrial redistribution of pS635-eNOS could be involved in the attenuation of inflammasome activity and the resolution of the inflammatory signal. This possibility is supported by data demonstrating that Aka is activated by protein nitration at Y350 in PAEC (R. Rafikov, J. Biol Chem, 288(9), 6212-26 (2013)).

The downstream effector of mitochondrial redistributed uncoupled eNOS is likely peroxynitrite, formed from the interaction of NO with superoxide. It has been shown that peroxynitrite levels in the lung increase in response to mechanical ventilation (L. Martinez-Caro, Shock, 44(1), 36-43 (2015); L. Martinez-Caro, Intensive Care Med, 35(6), 1110-9 (2009)). However, the protein targets are unresolved. Peroxynitrite introduces a covalent modification that adds a nitro group (—NO₂) to one ortho carbon of tyrosine's phenolic ring to form 3-nitrotyrosine (3-NT) in target proteins. Protein tyrosine nitration can alter the structure-function of affected proteins due to the introduction of a net negative charge to the nitrated tyrosine at physiological pH, (H. Gunaydin, Chem Res Toxicol, 22(5), 894-8 (2009)). Although this study did not identify the protein targets responsible for the disruption of mitochondrial bioenergetics and the increase in mitochondrial ROS, it is likely that at least one of these is carnitine acetyl transferase (CrAT) an important member of the carnitine shuttle involved in fatty acid oxidation (FAO). This is based on work which has identified CrAT as being susceptible to nitration mediated inhibition (S. Sharma, Lung Cellular and Molecular Physiology, 294(1), L46-56 (2008)) and identified the disruption of carnitine homeostasis as having a key role in the development of pulmonary vascular disease (S. Sharma, Lung Cellular and Molecular Physiology, 294(1), L46-56 (2008); S. Sharma, Pediatric Research, 74(1) 39-47 (2013); S. Sharma, Int J Mol Sci, 14(1), 255-72 (2012); X. Sun, Antioxidants & Redox Signaling, 18(14), 1739-52 (2013); S. Sharma, PLoS One, 7(9), e41555 (2012)). In addition, impaired FAO has been shown to be involved in the development of ALI (H. Cui, Am J Respir Cell Mol Biol, 167-178 (2019); 0. Kaya, Saudi Med J, 36(9), 1046-52 (2015); M. M. Sayed-Ahmed, J Egypt Natl Canc Inst, 16(4), 237-43 (2004)) and a pT495-eNOS mimic, T495D-eNOS induces CrAT nitration and disrupts carnitine homeostasis in PAEC (X. Sun, American Journal of Respiratory Cell and Molecular Biology, 50(6), 1084-95 (2014)).

In conclusion, the data establish a functional link between the activation of the mechanosensitive Ca²⁺ channel, TRPV4 and endothelial hyperpermeability through the phosphorylation and mitochondrial redistribution of eNOS mediated by PKC. The studies using an eNOS decoy peptide indicate that targeting mitochondrial dependent redox pathways may have significant therapeutic value in the treatment of VILI in humans.

Unless defined otherwise, all technical and scientific terms used herein have the same meanings as commonly understood by one of skill in the art to which the invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An isolated, synthetic peptide comprising from about 4 to 30 amino acids, wherein the peptide binds to Protein Kinase C (PKC) in vitro or in vivo.

2. The peptide of claim 1, comprising a PKC consensus binding sequence, wherein the consensus binding sequence comprises X-S/T-X-R/K (SEQ ID NO:5), wherein X is any amino acid.

3. The peptide of claim 2, wherein the consensus binding sequence comprises KTFK (SEQ ID NO:6).

4. The peptide of claim 2 further comprising from about 1-26 additional amino acids, optionally wherein the additional amino acids are comprised in a cell penetrating peptide.

5. The peptide of claim 1, wherein the peptide comprises the sequence HRKKRRQRRITRKKTFKEVA (SEQ ID NO:1) or ITRKKTFKEVA (SEQ ID NO:4), or a sequence comprising at least 70% sequence identity to HRKKRRQRRITRKKTFKEVA (SEQ ID NO:1) or ITRKKTFKEVA (SEQ ID NO:4).

6. The peptide of claim 1, wherein the peptide is further phosphorylated by Protein Kinase C (PKC), optionally wherein the peptide is phosphorylated at a threonine residue.

7. The peptide of claim 1, wherein exposure of the peptide to a cell reduces or prevents phosphorylation of endothelial nitric oxide synthase (eNOS), optionally wherein the phosphorylation of eNOS is at threonine 495 (T495).

8. The peptide of claim 1, wherein exposure of the peptide to a cell reduces or prevents redistribution/localization of eNOS to the mitochondria, production of NOS-derived superoxide, production of mitochondrial reactive oxygen species (ROS), loss of mitochondrial membrane potential, or combinations thereof.

9. The peptide of claim 1, wherein the PKC is PKCα.

10. The peptide of claim 7, wherein the cell is in a subject, preferably a human.

11. A pharmaceutical composition comprising the peptide of claim 1 and a pharmaceutically acceptable carrier.

12. The composition of claim 11 comprising a plurality of copies of the peptide.

13. A method of treating a subject having a disease, disorder, or condition comprising administering to the subject an effective amount of the composition of claim 11.

14. The method of claim 13, wherein the disease, disorder, or condition is associated with disruption of the endothelial barrier.

15. The method of claim 13, wherein the disease, disorder, or condition is selected from the group comprising pulmonary hypertension, gram positive sepsis, acute lung injury (ALI), ventilator-induced lung injury (VILI), chronic obstructive pulmonary disease (COPD), acute respiratory distress syndrome (ARDS), pulmonary fibrosis, systemic inflammatory response syndrome (SIRS), multiorgan dysfunction syndrome (MODS), COVID-19, and edema.

16. The method of claim 13, wherein the composition is administered in an effective amount to reduce or prevent inflammation or hypercytokinemia (cytokine storm) in the subject.

17. The method of claim 15, wherein the subject has ALI, VILI, or ARDS, and wherein the amount of the composition is effective to reduce vascular leakage or permeability, reduce bronchial alveolar lavage (BAL) protein levels, reduce BAL cell count, increase endothelial cell barrier integrity, reduce lung inflammation, or combinations thereof.

18. The method of claim 17, wherein the composition is administered prior to, during, or after mechanical ventilation of the subject.

19. The method of claim 13, wherein the composition is administered locally or systemically.

20. The method of claim 19, wherein the administration is by inhalation, intratracheal instillation, or intravenous administration.

21. The method of claim 13, wherein the subject is human.
